(19) United States
(12) Patent Application Publication
(10) Pub. No.: US 2026/0176313 A1
(43) Pub. Date: Jun. 25, 2026

(54) TGF-BETA-1 VACCINE

(71) Applicant: IO BIOTECH APS, Copenhagen (DK)

(72) Inventor: Mads Hald ANDERSEN, Naerum (DK)

(21) Appl. No.: 19/124,527

(22) PCT Filed: Nov. 3, 2023
(86) PCT No.: PCT/EP2023/080669

§ 371 Date: Apr. 25, 2025

(30) Foreign Application Priority Data
Nov. 4, 2022 (GB) 2216449.5

(51) Int. Cl. C07K 14/495; A61K 39/00; A61P 37/04

(52) U.S. Cl. CPC C07K 14/495; A61K 39/001134; A61P 37/04; A61K 2039/53; A61K 2039/55555

(57) ABSTRACT

The present invention relates to novel polypeptides, which are derived from transforming growth factor beta 1 (TGFβ1; TGFb-1) as well as polynucleotides encoding such polypeptides and compositions comprising such peptides. The present invention is further concerned with ways to increase the selectivity of the immune response to TGFb-1. The invention also concerns uses, and methods of using, said polypeptides, polynucleotides, and compositions.

### FIELD OF THE INVENTION

The present invention relates to novel polypeptides, which are derived from transforming growth factor beta 1 (TGFβ1; TGFb-1) as well as polynucleotides encoding such polypeptides and compositions comprising such peptides. The present invention is further concerned with ways to increase the selectivity of the immune response to TGFb-1. The invention also concerns uses, and methods of using, said polypeptides, polynucleotides, and compositions.

### BACKGROUND OF THE INVENTION

TGFb is a multifunctional cytokine with a key role in the regulation of the immune system. There are three isoforms, of which isoform1 (TGFb-1) is particularly important in T-cell immunity. In the context of cancer, TGFb-1 disarms various immune cells like cytotoxic T-cells (CTLs), tumor-associated neutrophils and Natural Killer (NK) cells. It also contributes to tumor vascularization and metastasis. Consequently, TGFb1 is a key inhibitory molecule in the tumor microenvironment (TME), contributing to a down-regulation of the immune system's anti-tumor machinery and enabling immune-evasion by cancer cells.

Recent clinical results (Kjeldsen, J. W. et al., Nat. Med. 27(12): 2212-2223 (2021)) provide a rationale for cancer immunotherapy based on activation of “anti-regulatory” T cells. Anti-regulatory T cells recognize antigens commonly expressed by immunosuppressive cells and thereby target pro-inflammatory signals to the tumor microenvironment (Andersen, M. H., Semin. Immunopathol (2022)). Therapeutics based on small molecule inhibitors targeting TGFb receptors and trap ligands based on soluble TGFb receptors target all three isoforms of TGFb (TGFb-1, TGFb-2, and TGFb-3) and the targeting of additional TGFb isoforms (TGFb-2, TGFb-3) has been suggested to contribute to adverse clinical effects (Tauriello, D. V. F., E. Sancho, and E. Batlle, Nat. Rev. Cancer, 22(1): 25-44 (2022)).

Activating TGFb-1-selective T cells (no cross-reactivity to TGFb-2 and TGFb-3) may allow targeting of pro-inflammatory immune response to TGFb-1-expressing tumors while avoiding the toxicity associated with pan-TGFb inhibition. TGFb-1-selective T cells are frequently detected in humans (Holmstrom, M. O., et al., Cell Mol. Immunol. 18(2): 415-426 (2021)). There is therefore an ongoing need to enhance the selectivity of anti-TGFb-1 immune responses to mitigate potential off-target toxicities.

### SUMMARY OF THE INVENTION

The present inventors have previously identified polypeptide fragments of TGFb-1 which are immunogenic. These polypeptide fragments are disclosed in WO 2020/245264, which is herein incorporated by reference. The present inventors have now identified new immunogenic polypeptide fragments from TGFb-1.

Surprisingly, the polypeptides disclosed herein stimulate an immune response which is selective for TGFb-1. In other words, the polypeptides disclosed herein stimulate TGFb-1 selective T cells which exhibit low cross reactivity towards TGFb-2 and TGFb-3. The polypeptides of the invention are therefore expected to be particularly effective at stimulating a beneficial, selective immune response against TGFb-1-expressing cells. In particular, the polypeptides of the present invention are expected to enhance the selectivity of the immune response against TGFb-1-expressing cells, without raising an immune response against TGFb-2-expressing cells or TGFb-3-expressing cells. The polypeptides of the present invention are therefore expected to display low off-target toxicity. The longer polypeptides of the present invention, such as those set forth in SEQ ID NOs: 7 and 10, are also expected to be particularly immunogenic as they may contain more epitopes than shorter polypeptide fragments of TGFb-1. Such peptides are also expected to have improved properties as regards ease of manufacture and formulation.

TGFb-1 is a dimeric cytokine which shares a cysteine knot structure connected together by intramolecular disulfide bonds. TGFb-1 is synthesized as a monomeric 390-amino acid precursor protein, which is referred to interchangeably as: TGFb-1 pre-protein; TGFb-1 precursor; full-length TGFb-1; pre-pro-TGFb-1. The full-length sequence of the TGFb-1 pre-protein is provided as SEQ ID NO: 1.

The TGFb-1 pre-protein monomer has a molecular weight of about 25 kDa. The TGFb-1 protein monomer has three distinct domains: the signal peptide (SP: amino acids 1-29; SEQ ID NO: 4), the latency associated peptide (LAP: amino acids 30-278; SEQ ID NO: 5) and the mature peptide (mature TGFb-1: amino acids 279-390; SEQ ID NO: 6).

The TGFb-1 SP targets the protein to a secretory pathway; the SP is cleaved off in the rough endoplasmic reticulum. TGFb-1 monomers comprising the LAP and mature TGFb-1 may dimerize in the endoplasmic reticulum via disulfide bridges between cysteine residues in the LAP (e.g. Cys 223 and Cys 225) and the mature TGFb-1 peptide (e.g. Cys 356) to form a TGFb-1 homodimer. This TGFb-1 homodimer is referred to as the small latent complex (SLC). The SLC may be bound by so-called latent TGF-β-Binding Protein (LTBP) to form a larger complex referred to as the large latent complex (LLC). The LLC may be secreted into extracellular media (ECM). However, the presence of LAP and the LTBP prevent TGFb-1 from binding to, and activating, its extracellular receptors. Active TGFb-1 consists of a homodimer of mature TGFb-1 peptides. There are various mechanisms by which the mature TGFb-1 homodimer is released from LAP and LTBP, which include degradation of LAP by proteases, induction of conformational change in LAP by interaction with thrombospondin, and rupture of noncovalent bonds between LAP and TGFb-1.

An object of the present invention is the development of an immunogenic polypeptide that raises a selective immune response against TGFb-1. Another object of the present invention is the selective targeting of immune suppressive cells in the TME. Given that TGFb-1 is highly expressed by immune suppressive cells in the TME, selectively targeting TGFb-1 on such cells therefore allows for the depletion of immune suppressive cells in the TME.

Thus, the present invention provides a polypeptide which is an immunogenic fragment of TGFb-1 and which comprises or consists of a sequence of at least 8 consecutive amino acids of SEQ ID NO: 5. Preferably, the polypeptide does not comprise a cysteine residue. The polypeptide may have low homology to a corresponding polypeptide sequence of TGFb-2 and/or TGFb-3. The polypeptide may have low sequence identity to a corresponding polypeptide sequence of TGFb-2 and/or TGFb-3. TGFb-2 may have the amino acid sequence of SEQ ID NO: 2. TGFb-3 may have the amino acid sequence of SEQ ID NO: 3. The polypeptide fragment may have less than about 80%, 70%, 60%, 50%, 40%, 30%, 25%, or 20% sequence identity to a corresponding polypeptide sequence of TGFb-2 and/or TGFb-3. Preferably, the polypeptide has less than about 40% sequence identity to a corresponding polypeptide sequence of TGFb-2 and/or TGFb-3. The polypeptide may comprise or consist of up to 8, 9, 10, 11, 12, 13, 14, 15, 16, 17, 18, 19, 20, 21, 22, 23, 24, 25, 26, 27, 28, 29, 30, 35, or 40 consecutive amino acids of SEQ ID NO: 5. The polypeptide may comprise or consist of at least 8, 9, 10, 11, 12, 13, 14, 15, 16, 17, 18, 19, 20, 21, 22, 23, 24, 25, 26, 27, 28, 29, 30, 35, or 40 consecutive amino acids of SEQ ID NO: 5. Preferably, the polypeptide may comprise or consist of at least 25, 26, 27, 28, 29, or 30 consecutive amino acids of SEQ ID NO: 5. More preferably, the polypeptide may comprise or consist of at least 30 consecutive amino acids of SEQ ID NO: 5. The polypeptide may comprise the amino acid sequence of SEQ ID NO: 32. The polypeptide may comprise the amino acid sequence of any one of SEQ ID NOs: 7, 10 or 11. The polypeptide may comprise or consist of the amino acid sequence of any one of SEQ ID NOs: 7, 10, 32, 11, 14, or 15. Preferably, the polypeptide comprises or consists of the amino acid sequence of SEQ ID NO: 7. Preferably, the polypeptide comprises or consists of the amino acid sequence of SEQ ID NO: 10. In one embodiment, the polypeptide does not comprise or consist of the amino acids sequence of SEQ ID NOs: 11, 14 and/or 15. In one embodiment, the polypeptide does not consist of the amino acid sequence of SEQ ID NOs: 11, 14 and/or 15.

The polypeptide may be capable of stimulating TGFb-1 selective T cells. The TGFb-1 selective T cells may have low cross reactivity towards cells expressing and/or presenting polypeptides of TGFb-2 and/or TGFb-3. Cells expressing and/or presenting polypeptides of TGFb-2 and/or TGFb-3 may be cells in the tumor microenvironment (TME). These cells may be tumor cells. Preferably, these cells are immune suppressive cells. The cells may include cancer associated fibroblasts (CAFs), CD8⁺ T cells, CD4⁺ T cells, regulatory CD4⁺ T cells, exhausted CD8⁺ T cells, M1 tumor associated macrophages (M1_TAM), M2 tumor associated macrophages (M2_TAM), myeloid antigen presenting cells (APCmye) and/or other cells.

The measurement of cross reactivity may comprise comparing the reactivity of the TGFb-1 selective T cells to cells expressing and/or presenting a polypeptide of TGFb-1 with the reactivity of the TGFb-1 selective T cells to cells expressing and/or presenting a corresponding polypeptide from TGFb-2 or TGFb-3. The reactivity of the TGFb-1 selective T cells towards cells expressing and/or presenting the corresponding polypeptide from TGFb-2 or TGFb-3 may be less than about 50%, 40%, 30%, 20%, 10%, 5%, 1%, or 0.1% of the reactivity of the TGFb-1 selective T cells to the polypeptide of TGFb-1. Cross reactivity may be measured by IFNγ ELISPOT assay.

The present invention further provides a polynucleotide encoding a polypeptide of the invention. The polynucleotide may be isolated. The polynucleotide may be comprised within a vector. The polynucleotide may be a messenger RNA (mRNA). The mRNA may comprise:
(a) an open reading frame (ORF) encoding at least one polypeptide of the invention;
(b) a 5′ terminal cap at the 5′ end;
(c) a 5′ untranslated region (UTR) which is included 5′ of the ORF;
(d) a 3′ UTR which is included 3′ of the ORF; and
(e) a 3′ tailing sequence at the 3′ end.

The present invention also provides a composition comprising a polypeptide of the invention and/or a polynucleotide of the invention and optionally an adjuvant. The composition may further comprise at least one different polypeptide of the invention; at least one different polynucleotide of the invention; and/or at least one pharmaceutically acceptable diluent, carrier or preservative. The adjuvant may be selected from the group consisting of bacterial DNA based adjuvants, oil/surfactant based adjuvants, viral dsRNA based adjuvants, imidazoquinolines, and a Montanide ISA adjuvant. Preferably, the composition may be a TGFb-1 selective vaccine composition. Where the composition comprises a polynucleotide and the polynucleotide is mRNA, the composition may be formulated in a lipid nanoparticle composition. The lipid nanoparticle composition may have a mean diameter of 50-200 nm.

The present invention also provides a method of treating or preventing a disease or condition in a subject, the method comprising administering to the subject a polypeptide of the invention, a polynucleotide of the invention, and/or a composition of the invention. The method may further comprise the simultaneous or sequential administration of an additional cancer therapy, preferably an antibody. Where the disease or condition is cancer, the method may further comprise stimulation of a selective immune response to TGFb-1-expressing cancer cells.

The present invention also provides a polypeptide of the invention, a polynucleotide of the invention, a composition of the invention, or a combination thereof for use in treating or preventing a disease or condition. The polypeptide, polynucleotide, composition, or combination thereof may be for use in combination with an additional cancer therapy, preferably an antibody. Where the disease or condition is cancer, the polypeptide, polynucleotide, composition, or combination thereof may stimulate a selective immune response to TGFb-1-expressing cancer cells.

The present invention further provides use of a polypeptide of the invention, a polynucleotide of the invention, a composition of the invention, or a combination thereof for the manufacture of a medicament for the treatment or prevention of a disease or condition. Where the disease or condition is cancer, the use of the polypeptide, polynucleotide, composition, or combination thereof, may be for the manufacture of a medicament for stimulating a selective immune response to TGFb-1-expressing cancer cells.

The disease or condition may be characterized at least in part by inappropriate or excessive immune suppressive function of TGFb1-expressing cells. Preferably, the disease or condition is a cancer. Said cancer may be an esophageal cancer or a urothelial cancer. Said cancer may be a colorectal carcinoma, a gastric cancer, a head and neck cancer, a melanoma, a non-small-cell lung carcinoma (NSCLC) or an ovarian cancer. Said cancer may be a breast cancer, a cervical cancer, a liver cancer, or a pancreatic cancer.

The disease or condition may be a tumor and the polypeptide, polynucleotide, composition, or combination thereof, may be capable of modulating the tumor microenvironment (TME). The methods and uses of the invention may comprise modulating the TME. The modulating may comprise enhancing infiltration of the TME by T cells. Preferably, the T cells are CD4⁺ T cells. Administering the polypeptide, polynucleotide, composition, or a combination thereof, may stimulate a selective immune response to TGFb-1-expressing cells in the TME.

The present invention also provides a method of stimulating TGFb-1 selective T cells, the method comprising contacting the T cells with a polypeptide of the invention, a polynucleotide of the invention, a composition of the invention, or a combination thereof.

The present invention also provides an ex vivo method of stimulating TGFb-1 selective T cells, the method comprising contacting the T cells with a polypeptide of the invention, a polynucleotide of the invention, a composition of the invention, or a combination thereof.

The present invention further provides the use of a polypeptide of the invention, a polynucleotide of the invention, a composition of the invention, or a combination thereof for stimulating TGFb-1 selective T cells. The use may comprise contacting the T cells with a polypeptide of the invention, a polynucleotide of the invention, a composition of the invention, or a combination thereof. The use may be non-therapeutic and/or ex vivo.

The TGFb-1 selective T cells stimulated by the peptides, polynucleotides and/or compositions of the present invention may have low cross reactivity towards cells expressing and/or presenting polypeptides of TGFb-2 and/or TGFb-3. Measurement of cross reactivity may comprise comparing the reactivity of the TGFb-1 selective T cells to cells expressing and/or presenting a polypeptide of TGFb-1 with the reactivity of the TGFb-1 selective T cells to cells expressing and/or presenting a corresponding polypeptide from TGFb-2 or TGFb-3. The reactivity of the TGFb-1 selective T cells towards cells expressing and/or presenting the corresponding polypeptide from TGFb-2 or TGFb-3 may be less than about 50%, 40%, 30%, 20%, 10%, 5%, 1%, or 0.1% of the reactivity of the TGFb-1 selective T cells to the polypeptide of TGFb-1. Cross reactivity may be measured by IFNγ ELISPOT assay.

### BRIEF DESCRIPTION OF THE FIGURES

FIG. 1A-B. Representative images showing TGFB-1 staining on tumor cells in esophageal cancer (A) and non-small cell lung cancer (NSCLC) (B).

FIG. 2. TGFb-1 is expressed by a substantial fraction of cells in tumors and the tumor microenvironment (TME). Boxplots and datapoints show the frequency of TGFb-1 expression in tumor cells and the tumor microenvironment (TME, not tumor cells) of the indicated cancer types. For each cancer type, boxplots on the left hand side correspond to TGFb-1 expression in the TME, whereas boxplots on the right hand side correspond to TGFb-1 expression in tumor cells. The fraction of TGFb-1 positive cells in each individual region of interest (ROI) is shown as a single datapoint.

FIG. 3. TGFb-1 is expressed by various cell types in the TME. The column charts show the fraction of TGFb-1 positive cells of each cell type in the TME (tumor cells excluded) of the indicated cancer types. Some categories are non-exclusive, so the total can exceed 1.0. The order of cell types presented in the chart key is identical to the order of cell types in each column.

FIG. 4. TGFb-1 expressing cells are not confined to the TME population expressing other immune suppression markers. The column chart shows the fraction of cells expressing the indicated immunosuppressive antigens. “Combination” refers to cells expressing any combination of two or more antigens. The melanoma samples analyzed here are 70% uveal and 30% cutaneous, metastatic melanoma was not analyzed. The order of immune suppression markers presented the chart key is identical to the order of immune suppression markers in each column.

FIG. 5. TGFb sequence alignment. Clustal Omega sequence alignment of the human TGFb-1, TGFb-2, and TGFb-3 proteins. The labels Pep01 to Pep12 in FIG. 5 indicate the positions of the peptides disclosed herein mapped on to the sequences of TGFb-1, TGFb-2 and TGFb-3. For example, the sequence of Pep01-1 can be found in the sequence of TGFb-1 underneath “Pep01” in FIG. 5, the sequence of Pep01-2 can be found in the sequence of TGFb-2 underneath “Pep01” in Figure, and so on.

FIG. 6. IFNγ ELISPOT identifies strong and frequent immune responses. Immune responses to TGFb-1 peptides were identified in PBMCs from 14 healthy donors. PBMCs were stimulated with the indicated TGFb-1 peptides and responses were analyzed after seven days by IFNγ ELISPOT. Significant responses (*) were defined by a Fisher exact P value<0.01, a ratio of peptide to control spots>2, and background subtracted spots>25. Peptides that elicited responses in the greatest number of donors were subsequently tested for the specificity of the response to TGFb-1.

FIG. 7A-B. IFNγ ELISPOT to identify TGFb-1-selective immune responses. Donor PBMCs were stimulated with the indicated TGFb-1 peptides (Pep01-1, Pep04-1, Pep05-1, Pep08-1, Pep09-1 and Pep12-1) and after seven days were tested for a recall response by IFNγ ELISPOT assay using the same TGFb-1 peptide, or homologous TGFb-2 peptide (i.e. Pep01-2, Pep04-2, Pep05-2, Pep08-2, Pep09-2 or Pep12-2) or TGFb-3 peptide (i.e. Pep01-3, Pep04-3, Pep05-3, Pep08-3, Pep09-3 or Pep12-3). The highly homologous peptides Pep12-1, Pep12-2 and Pep12-3 were included as positive controls for detecting cross-reactive immune responses to TGFb-2 and TGFb-3. In FIG. 7A, for each peptide tested, boxplots on the left hand side correspond to responses using the same TGFb-1 peptide, boxplots in the middle correspond to responses using homologous TGFb-2 peptides and boxplots on the right hand side correspond to responses using homologous TGFb-3 peptides. FIG. 7B shows representative ELISPOT assays, set up in triplicate wells.

FIG. 8. IFNγ ELISPOT to identify TGFb-1-selective immune responses. The same data shown in FIG. 7 were plotted to show responses for individual peptides and donors. Significant cross-reactive responses to homologous TGFb-2 and TGFb-3 peptides were observed only once (Pep01-2), while cross reactivity was not observed in 7 other donors tested with this peptide combination. The highly homologous peptide Pep12 displayed cross-reactivity in all donors that exhibited significant responses to the TGFb-1 homolog (Pep12-1). Significant responses (*) were defined by a Fisher exact P value<0.01, a ratio of peptide to control spots>2, and background subtracted spots>25.

FIG. 9. TGFb-1 vaccine induces robust immune responses. Mice were vaccinated with two different synthetic long peptides (SLPs) and immune responses were analyzed by IFNγ ELISPOT assay. ELISPOT responses for individual mice are shown. Results for control peptides are clearly indicated.

FIG. 10A-D. TGFb-1 vaccine drives derived immune permissive changes in the TME. Tumor weight was compared between groups at experiment termination (day 21). TGFB-1 expression was assayed by latency associated peptide (LAP) staining. CD4⁺ T cell infiltration was significantly enhanced in SLP2 vaccinated animals while CD8⁺ T cell infiltration remained unchanged.

FIG. 11. TGFb-1 vaccine promotes targeted in vivo cell killing. An in vivo cytotoxicity assay was used to compare cell killing in mice vaccinated with SLP1 versus the class I epitope SLP1_Ib (SIYMFFNT). Vaccinated mice (duplicates) were injected with differentially labeled splenocytes loaded with the assay peptide (SLP1_Ib) or a control peptide. Cell killing was determined by comparing the recovery of the splenocytes using control-peptide-loaded splenocytes as an internal control for cell recovery. OVA peptide was included as a positive control.

### BRIEF DESCRIPTION OF THE SEQUENCES

SEQ ID NO: 1 is the amino acid sequence of the full-length precursor of human TGFb-1 (also referred to as the TGFb-1 pre-protein).
SEQ ID NO: 2 is the amino acid sequence of the full-length precursor of human TGFb-2 (also referred to as the TGFb-2 pre-protein).
SEQ ID NO: 3 is the amino acid sequence of the full-length precursor of human TGFb-3 (also referred to as the TGFb-3 pre-protein).
SEQ ID NO: 4 is the signal peptide of human TGFb-1.
SEQ ID NO: 5 is the amino acid sequence of the latency associated peptide (LAP) domain of human TGFb-1.
SEQ ID NO: 6 is the amino acid sequence of mature human TGFb-1.
SEQ ID NOs: 7-17 are each an amino acid sequence of a polypeptide fragment derived from human TGFb-1.
SEQ ID NOs: 18-22 are each an amino acid sequence of a polypeptide fragment derived from human TGFb-2.
SEQ ID NOs: 23-27 are each an amino acid sequence of a polypeptide fragment derived from human TGFb-2.
SEQ ID NO: 28 is an amino acid sequence of a polypeptide fragment derived from human TGFb-1 that shares sequence homology with human TGFb-2 and TGFb-3.
SEQ ID NO: 29 is an amino acid sequence of a polypeptide fragment derived from human TGFb-2 that shares sequence homology with human TGFb-1 and TGFb-3.
SEQ ID NO: 30 is an amino acid sequence of a polypeptide fragment derived from human TGFb-3 that shares sequence homology with human TGFb-1 and TGFb-2.
SEQ ID NOs: 31 and 34 are synthetic long peptides (SLPs) comprising predicted MHC class I and class II epitopes.
SEQ ID NOs: 32, 33, 35, and 36 are minimal peptides corresponding to MHC class I and class II epitopes of the SLPs.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular (“a”, “an”, and “the”) will also include the plural and vice versa unless the content clearly dictates otherwise. Thus, for example, reference to “a polypeptide” includes “polypeptides”, and the like. In the event that any description of a term set forth conflicts with any document incorporated herein by reference, the description of the term set forth below shall control.

In instances where the terms “comprising” and “comprises” are used, also provided is something “consisting essentially of” or “consisting of” what is set out.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

The terms “polynucleotide”, “nucleic acid” and “nucleic acid molecule” are used interchangeably herein and refer to a polymeric form of nucleotides of any length, either deoxyribonucleotides or ribonucleotides, or analogs thereof.

The terms “patient” and “subject” are used interchangeably and typically refer to a human.

By “immunogenic” herein it is meant that a polypeptide is capable of eliciting an immune response to the TGFb protein, in particular TGFb-1 protein, typically when said protein is present in or on cells expressing the TGFb-1 protein. In other words, the polypeptide may be described as immunogenic to TGFb. The polypeptide may alternatively be described as an immunogenic fragment of TGFb. The immune response may refer to a T cell response, and so the polypeptide may be described as an immunogenic fragment of TGFb comprising a T cell epitope. The immune response may be detected in at least one individual (or in sample taken from the individual) after administration of the polypeptide to said individual (or said sample).

A polypeptide may be identified as immunogenic using any suitable method, including in vitro methods. For example, a peptide may be identified as immunogenic if it has at least one of the following characteristics:
i. it is capable of eliciting IFN-γ-producing cells in a PBL population of a healthy subject and/or a cancer patient as determined by an ELISPOT assay; and/or
ii. it is capable of in situ detection in a sample of tumor tissue of CTLs that are reactive with TGFb-1; and/or
iii. it is capable of inducing the in vitro growth of specific T-cells.

Methods suitable for determining whether a polypeptide is immunogenic are also described in the Examples section below.

The polypeptides disclosed here may be capable of stimulating a “selective” immune response to TGFb-1, such as a selective T cell response to TGFb-1. In this context, a selective immune response to TGFb-1 is considered to be one which is greater than an immune response to TGFb-2 or TGFb-3. For example, a polypeptide may be considered capable of stimulating a selective T cell response to TGFb-1 provided that said polypeptide does not stimulate a T cell response to TGFb-2 and/or TGFb-3. Similarly, a polypeptide may be considered capable of stimulating a selective T cell response to TGFb-1 provided that said polypeptide stimulates a T cell response to TGFb-1 that is at least about 2-fold, 5-fold, 10-fold, 100-fold, 1000-fold, 10 000-fold higher than the T cell response to TGFb-2 and/or TGFb-3 that said polypeptide is capable of stimulating.

Suitable assays for measuring a selective immune response to TGFb-1 would be apparent to persons skilled in art. An exemplary assay that may be used for this purpose is IFNγ ELISPOT assay. For example, a polypeptide may be identified as being capable of stimulating a selective immune response to TGFb-1 if:
i. it is capable of eliciting IFNγ-producing cells in a peripheral blood leukocyte (PBL) population of a healthy subject and/or a cancer patient as determined by an ELISPOT assay; and
ii. the IFNγ-producing cells, when contacted with a corresponding polypeptide from TGFb-2 or TGFb-3, produces less IFNγ as determined by an ELISPOT assay.

Reference herein to “TGFb,” “TGF-b”, “T-GF-beta”, and the like corresponds to reference to TGF-β. However, to avoid the use of Greek symbols and aid reproducibility of the text, the former nomenclature has been used.

### Polypeptides

In any polypeptide described herein, the amino acid sequence may be modified by one, two, three, four, or five (that is up to five) additions, deletions or substitutions, provided that a polypeptide having the modified sequence exhibits the same or increased immunogenicity to TGFb1, as compared to a polypeptide having the unmodified sequence. By “the same” it is to be understood that the polypeptide of the modified sequence does not exhibit significantly reduced immunogenicity to TGFb1 as compared to polypeptide of the unmodified sequence. Any comparison of immunogenicity between sequences is to be conducted using the same assay. Unless otherwise specified, modifications to a polypeptide sequence are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A1 below. Where amino acids have similar polarity, this can be determined by reference to the hydropathy scale for amino acid side chains in Table A2.

TABLE A1
Chemical properties of amino acids

| | | | |
|---|---|---|---|
| Ala (A) | aliphatic, hydrophobic, neutral | Met (M) | hydrophobic, neutral |
| Cys (C) | polar, hydrophobic, neutral | Asn (N) | polar, hydrophilic, neutral |
| Asp (D) | polar, hydrophilic, charged (−) | Pro (P) | hydrophobic, neutral |
| Glu (E) | polar, hydrophilic, charged (−) | Gln (Q) | polar, hydrophilic, neutral |
| Phe (F) | aromatic, hydrophobic, neutral | Arg (R) | polar, hydrophilic, charged (+) |
| Gly (G) | aliphatic, neutral | Ser (S) | polar, hydrophilic, neutral |
| His (H) | aromatic, polar, hydrophilic, charged (+) | Thr (T) | polar, hydrophilic, neutral |
| Ile (I) | aliphatic, hydrophobic, neutral | Val (V) | aliphatic, hydrophobic, neutral |
| Lys (K) | polar, hydrophilic, charged(+) | Trp (W) | aromatic, hydrophobic, neutral |
| Leu (L) | aliphatic, hydrophobic, neutral | Tyr (Y) | aromatic, polar, hydrophobic |

TABLE A2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

In any polypeptide disclosed herein, any one or more of the following modifications may be made to improve physiochemical properties (e.g. stability), provided that the polypeptide exhibits the same or increased immunogenicity to TGFb1, as compared to a polypeptide having the unmodified sequence:

Replacement of the C terminal amino acid with the corresponding amide (may increase resistance to carboxypeptidases);

Replacement of the N terminal amino acid with the corresponding acylated amino acid (may increase resistance to aminopeptidases);

Replacement of one or more amino acids with the corresponding methylated amino acids (may improve proteolytic resistance); and/or

Replacement of one or more amino acids with the corresponding amino acid in D-configuration (may improve proteolytic resistance).

Any polypeptide disclosed herein may have attached at the N and/or C terminus at least one additional moiety to improve solubility, stability and/or to aid with manufacture/isolation, provided that the polypeptide exhibits the same or increased immunogenicity to TGFb1, as compared to a polypeptide lacking the additional moiety. Suitable moieties include hydrophilic amino acids. For example, the amino acid sequences KK, KR or RR may be added at the N terminus and/or C terminus. Other suitable moieties include Albumin or PEG (Polyethylene Glycol).

A polypeptide as disclosed herein may be produced by any suitable means. For example, the polypeptide may be synthesised directly using standard techniques known in the art, such as Fmoc solid phase chemistry, Boc solid phase chemistry or by solution phase peptide synthesis. Alternatively, a polypeptide may be produced by transforming a cell with a nucleic acid molecule or vector which encodes said polypeptide. Such cells typically include prokaryotic cells such as bacterial cells, for example E. coli. Such cells may be cultured using routine methods to produce a polypeptide of the invention. The invention provides nucleic acid molecules and vectors which encode a polypeptide of the invention. The invention also provides a host cell comprising such a nucleic acid or vector.

The polypeptide of the invention may be in a substantially isolated form. It may be mixed with carriers, preservatives, or diluents which will not interfere with the intended use, and/or with an adjuvant and still be regarded as substantially isolated. It may also be in a substantially purified form, in which case it will generally comprise at least 90%, e.g. at least 95%, 98% or 99%, of the protein in the preparation.

For the purpose of this invention, in order to determine the percent identity of two sequences (such as two polypeptide sequences), the sequences are aligned for optimal comparison purposes (e.g. gaps can be introduced in a first sequence for optimal alignment with a second sequence). The nucleotides at each position are then compared. When a position in the first sequence is occupied by the same nucleotide as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is less than 80% identical to SEQ ID NO: 18, SEQ ID NO: 18 would be the reference sequence. To assess whether a sequence is less 80% identical to SEQ ID NO: 18 (an example of a reference sequence), the skilled person would carry out an alignment over the length of SEQ ID NO: 18, and identify how many positions in the test sequence were identical to those of SEQ ID NO: 18. If less than 80% of the positions are identical, the test sequence is less than 80% identical to SEQ ID NO: 18. If the sequence is shorter than SEQ ID NO: 18, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

### Polynucleotides

Non-limiting examples of polynucleotides of the present invention include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press). The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al. (1989, Molecular Cloning—a laboratory manual; Cold Spring Harbor Press).

In one embodiment, the polynucleotide is an mRNA. The mRNA may comprise:
a) an open reading frame (ORF) encoding at least one immunogenic polypeptide of the invention;
b) a 5′ terminal cap at the 5′ end;
c) a 5′ untranslated region (UTR) which is included 5′ of the ORF;
d) a 3′ UTR which is included 3′ of the ORF; and
e) a 3′ tailing sequence at the 3′ end.

In the mRNA sequences encoding an immunogenic polypeptide may each be interspersed by a cleavage sensitive site.

The ORF may include multiple copies of each sequence encoding a different immunogenic polypeptide, optionally at least 2, 3, 4, 5, 10, 15, 20, 25, 30, 35, 40, 45, 50 or more 15 copies of each said sequence, and preferably wherein the ORF encodes at least 2, 3, 4, 5, 10 or more different immunogenic polypeptides.

The mRNA may be described as a mRNA vaccine against cancer, or a mRNA cancer vaccine. mRNA vaccines are described in International Patent Application No. WO 2015/164674, which is herein incorporated by reference in its entirety. The mRNA cancer vaccines of the invention may be compositions, including pharmaceutical compositions. The invention also encompasses methods for the preparation, manufacture, formulation, and/or use of mRNA cancer vaccines.

The fact that the immunogenic polypeptides are expressed from RNA as intracellular peptides may provide advantages over delivery as exogenous peptides. The RNA is delivered intra-cellularly and expresses the epitopes in proximity to the appropriate cellular machinery for processing the epitopes such that they will be recognized by the appropriate immune cells. Additionally, a targeting sequence may allow more specificity in the delivery of the peptide epitopes. For example, a C-terminus Ubiquitin Ligase targeting protein (FBox Protein) may be used to target the polypeptide processing to the proteasome and more closely mimic the MHC processing. The constructs of the invention also may include linkers such as proteolytic cleavage sites optimized for APCs. These proteolytic sites provide an advantage because they enhance the processing of the peptides in APCs. When the mRNA cancer vaccine is delivered to a cell, the mRNA will be processed into a polypeptide by the intracellular machinery which can then process the polypeptide into the immunogenic polypeptides capable of stimulating a desired immune response.

In some embodiments, the mRNA cancer vaccine encodes multiple immunogenic polypeptides. This may be described as a poly-epitopic mRNA vaccine because each encoded immunogenic polypeptide comprises at least one epitope. The RNA sequences that code for the immunogenic polypeptides may be interspersed by sequences that code for amino acid sequences recognized by proteolytic enzymes. Thus, in some embodiments an mRNA cancer vaccine is an mRNA having an open reading frame encoding a propeptide, since the encoded polypeptide sequence includes multiple immunogenic polypeptides linked together either directly or through a linker such as a cleavage sensitive site. An exemplary propeptide has the following peptide sequence:

$$Tm - Yo - ( X 1 - Yo - X 2 - Yo - … . Xn ) - Yo - Tm$$

Where: T is a targeting sequence and m=0-1. A targeting sequence may be included at either the N terminus, the C terminus, or both ends of the central peptide region. If a polypeptide has more than one targeting sequence, those sequences may be the same or different.

X1, 2 etc. are each independently an immunogenic polypeptide sequence, and n=0-1000. Each immunogenic polypeptide sequence designated by an X may represent a unique immunogenic polypeptide sequence in the propeptide or it may refer to a copy of a immunogenic polypeptide sequence. Thus, the propeptide encoded by the mRNA may be composed of multiple immunogenic polypeptide sequences each of which is unique and/or it may include more than 1 copy of each unique immunogenic polypeptide sequence(s). In some embodiments a propeptide may have at least 2, at least 3, at least 4, at least 5, at least 10, at least 20, at least 30, at least 40, at least 50, or more copies of each unique immunogenic polypeptide sequence. Preferably the propeptide has at least 2, 3, 4, 5, 10 or more different immunogenic polypeptide sequences.

Y is a linker sequence, preferably a cleavage sensitive sequence, and o=0-5. Each immunogenic polypeptide sequence may optionally have one or more linkers, optionally cleavage sensitive sites adjacent to the N and/or C terminal end. In a multiepitope design, two or more of the immunogenic polypeptide sequences may have a cleavage sensitive site between them. Alternatively two or more of the immunogenic polypeptide sequences may be connected directly to one another or through a linker that is not a cleavage sensitive site. The targeting sequence may also be connected to the immunogenic polypeptide sequence through a cleavage sensitive site or it may be connected directly to the immunogenic polypeptide sequence through a linker that is not a cleavage sensitive site.

The mRNA may encode one or more targeting sequence. This may be an endosomal targeting sequence, for example a portion of the transmembrane domain of lysosome associated membrane protein (LAMP-1) or a portion of the transmembrane domain of invariant chain (Ii). The targeting sequence may be a ubiquitination signal that is attached at either or both ends of the encoded polypeptide. In other embodiments, the targeting sequence is a ubiquitination signal that is attached at an internal site of the encoded polypeptide and/or to either end. Thus, the RNA may include a nucleic acid sequence encoding a ubiquitination signal at either or both ends of the nucleotides encoding the immunogenic polypeptide(s).

Ubiquitination, a post-translational modification, is the process of attaching ubiquitin to a substrate target protein. A ubiquitination signal is a peptide sequence which enables the targeting and processing of a peptide to one or more proteasomes. By targeting and processing the peptide through the use of a ubiquitination signal the intracellular processing of the peptide can more closely recapitulate antigen processing in Antigen Presenting Cells (APCs). The number of ubiquitins added to an antigen can enhance the efficacy of the processing step. For instance, in polyubiquitination, additional ubiquitin molecules are added after the first has been attached to the peptide. The resulting ubiquitin chain is created by the linking of the glycine residue of the ubiquitin molecule to a lysine of the ubiquitin bound to the peptide. Each ubiquitin contains seven lysine residues and an N-terminal that can serve as sites for ubiquitination. When four or more ubiquitin molecules are attached to a lysine residue on the peptide antigen, the 26S proteasome recognizes the complex, internalizes it, and degrades the protein into small peptides.

The immunogenic polypeptide sequences may be connected in some embodiments by a cleavage sensitive site. A cleavage sensitive site is a peptide which is susceptible to cleavage by an enzyme or protease. These sites are also called protease cleavage sites. Preferably the protease is an intracellular enzyme. The protease may be a serine protease, a threonine protease, a cysteine protease, an aspartate protease, a glutamic acid protease, or a metalloprotease. In some preferred embodiments the protease is a protease found in an Antigen Presenting Cell (APC). Thus, protease cleavage sites correspond to high abundance (highly expressed) proteases in APCs. A cleavage sensitive site that is sensitive to an APC enzyme is referred to as an APC cleavage sensitive site. Proteases expressed in APCs include but are not limited to Cysteine proteases, such as: Cathepsin B, Cathepsin H, Cathepsin L, Cathepsin S, Cathepsin F, Cathepsin Z, Cathepsin V, Cathepsin O, Cathepsin C, and Cathepsin K, and Aspartic proteases such as Cathepsin D, Cathepsin E, and Asparaginyl endopeptidase.

The cleavage sensitive site may preferably be a cathepsin B or S sensitive site. Exemplary cathepsin B sensitive sites include but are not limited to those described in WO 2017/020026 (which is herein incorporated by reference; see SEQ ID NOs: 12 to 407 of WO 2017/020026). Exemplary cathepsin S sensitive sites include but are not limited to those described in WO 2017/020026 (see SEQ ID NOs: 3 to 5, 408 to 1122 of WO 2017/020026). Other cathepsin sensitive sites are known in the art or can easily be determined experimentally using digestion assays with no more than routine experimentation.

The mRNA cancer vaccines may comprise one or more polynucleotides, which encode one or more immunogenic polypeptide sequences of the invention. Exemplary polynucleotides may include at least one chemical modification. The polynucleotides can include various substitutions and/or insertions. As used herein in a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population.

A modified polynucleotide may, when introduced to a cell or organism, exhibit reduced degradation in the cell or organism, as compared to an unmodified polynucleotide. A modified polynucleotide may, when introduced into a cell or organism, exhibit reduced immunogenicity in the cell or organism (e.g., a reduced innate response.). Modifications of polynucleotides are well known in the art and include, for example, those listed in WO 2017/020026. Generally, the modifications discussed in this section are not intended to refer to the ribonucleotide modifications in naturally occurring 5′-terminal mRNA cap moieties.

The polynucleotide may comprise modifications which are naturally occurring, non-naturally occurring or the polynucleotide can comprise both naturally and non-naturally occurring modifications. The polynucleotides of the mRNA cancer vaccines of the invention can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein. Non-natural modified nucleotides may be introduced to polynucleotides, e.g., of the mRNA cancer vaccines, or nucleic acids during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme. Any of the regions of the polynucleotides may be chemically modified.

The present disclosure provides for modified nucleosides and nucleotides. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphodiester linkages, in which case the polynucleotides would comprise regions of nucleotides.

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into the polynucleotides of the invention.

The mRNA may at least one chemical modification, which is preferably selected from pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-seudouridine, 5-aza-uridine, dihydropseudouridine, 5-methyluridine, 5-methoxyuridine, and 2′-0-methyl uridine.

As used herein, “messenger RNA” (mRNA) refers to any polynucleotide which encodes at least one peptide or polypeptide of interest and which is capable of being translated to produce the encoded peptide polypeptide of interest in vitro, in vivo, in situ or ex vivo. The basic components of an mRNA molecule include at least a coding region, a 5′UTR, a 3′UTR, a 5′ terminal cap and a 3′ tailing sequence. The mRNA of the invention typically includes all of these features.

A “5′ untranslated region (UTR)” is a region of an mRNA that is directly upstream (i.e., 5′) from the start codon (i.e., the first codon of an mRNA transcript translated by a ribosome) that does not encode a protein or peptide.

A “3′ untranslated region (UTR)” is a region of an mRNA that is directly downstream (i.e., 3′) from the stop codon (i.e., the codon of an mRNA transcript that signals a termination of translation) that does not encode a protein or peptide.

An “open reading frame” is a continuous stretch of DNA beginning with a start codon (e.g., methionine (ATG)), and ending with a stop codon (e.g., TAA, TAG or TGA) and encodes a protein or peptide.

A 5′ terminal cap is a specially altered nucleotide on the 5′ end of some primary transcripts such as messenger RNA, which promotes stability and translation. It usually consists of a guanine nucleotide connected to mRNA via an unusual 5′ to 5′ triphosphate linkage. This guanosine is methylated on the 7 position directly after capping in vivo by a methyltransferase. It may thus be referred to as a 7-methylguanylate cap, abbreviated as m7G. A preferred 5′ terminal cap is m7G (5′)ppp(5′)N1mpNp.

The 3′ tailing sequence is a polyA tail, a polyA-G quartet and/or a stem loop sequence. The 3′ tailing sequence is typically between 40 and 200 nucleotides in length. In some embodiments, the 3′ tailing sequence is a polyA tail. A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

In some embodiments, the polynucleotide includes from about 200 to about 3,000 nucleotides (e.g., from 200 to 500, from 200 to 1,000, from 200 to 1,500, from 200 to 3,000, from 500 to 1,000, from 500 to 1,500, from 500 to 2,000, from 500 to 3,000, from 1,000 to 1,500, from 1,000 to 2,000, from 1,000 to 3,000, from 1,500 to 3,000, and from 2,000 to 3,000).

The polynucleotides of the present invention may function as mRNA but are distinguished from wild-type mRNA in functional and/or structural features. mRNA cancer vaccines of the present invention may be encoded by in vitro translated (IVT) polynucleotides. An “in vitro transcription template (IVT),” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

mRNA may be prepared by any suitable technique known in the art, via any appropriate synthesis route. IVT methods are preferred. In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities) (Beckert et ah, Synthesis of RNA by in vitro transcription, Methods Mol Biol. 703:29-41(2011); Rio et al. RNA: A Laboratory Manual. Cold Spring Harbor: Cold Spring Harbor Laboratory Press, 2011, 205-220.; Cooper, Geoffery M. The Cell: A Molecular Approach. 4th ed. Washington D.C.: ASM Press, 2007. 262-299). Generally, IVT utilizes a DNA template featuring a promoter sequence upstream of a sequence of interest. The promoter sequence is most commonly of bacteriophage origin (ex. the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand. Suitable methods include, for example, those listed in WO2017/020026 (which is herein incorporated by reference).

The mRNA disclosed herein may be in whole or in part codon optimized for human expression and/or for reducing immune recognition. Codon optimization methods are known in the art and may be useful in efforts to achieve various results, such as to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park CA) and/or proprietary methods. Preferably, the ORF sequence is optimized using optimization algorithms.

A codon optimized sequence may share less than 95%, 90%, 85%, 80%, or 75% sequence identity to a naturally-occurring or wild-type sequence (e.g., a naturally-occurring or wild-type mRNA sequence encoding a polypeptide or protein of interest. A codon optimized sequence may share between 65% and 85% sequence identity to a naturally-occurring or wild-type sequence (e.g., a naturally-occurring or wild-type mRNA sequence encoding a polypeptide or protein of interest.

An exemplary codon optimized RNA may be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 (herein incorporated by reference) discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Compositions, Formulations, Encapsulations

The present invention provides a composition comprising a polypeptide of the invention and/or a polynucleotide of the invention. For example, the invention provides a composition comprising one or more polypeptides of the invention and/or one or more polynucleotides of the invention, and optionally at least one adjuvant, pharmaceutically acceptable carrier, preservative and/or excipient.

The composition may comprise at least two, at least three, at least four, at least five, at least six, at least seven, at least eight different polypeptides of the invention and optionally at least one adjuvant, pharmaceutically acceptable carrier, preservative and/or excipient.

The composition may comprise at least two, at least three, at least four, at least five, at least six, at least seven, at least eight different polynucleotides of the invention and optionally at least one adjuvant, pharmaceutically acceptable carrier, preservative and/or excipient.

The carrier, preservative and excipient must be ‘acceptable’ in the sense of being compatible with the other ingredients of the composition and not deleterious to a subject to which the composition is administered. Typically, all components and the final composition are sterile and pyrogen free.

The composition may be a pharmaceutical composition.

The composition may be a vaccine composition, preferably a TGFb-1 selective vaccine composition.

The composition may preferably comprise an adjuvant. Adjuvants are any substance whose admixture into the composition increases or otherwise modifies the immune response elicited by the composition. Adjuvants, broadly defined, are substances which promote immune responses. Adjuvants may also preferably have a depot effect, in that they also result in a slow and sustained release of an active agent from the administration site. A general discussion of adjuvants is provided in Goding, Monoclonal Antibodies: Principles & Practice (2nd edition, 1986) at pages 61-63.

Adjuvants may be selected from the group consisting of: AlK(SO₄)₂, AlNa(SO₄)₂, AlNH₄(SO₄), silica, alum, Al(OH)₃, Ca₃(PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, Titermax, ISCOMS, Quil A, ALUN (see U.S. Pat. No. 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Various saponin extracts have also been suggested to be useful as adjuvants in immunogenic compositions. Granulocyte-macrophage colony stimulating factor (GM-CSF) may also be used as an adjuvant.

Preferred adjuvants to be used with the invention include oil/surfactant based adjuvants such as Montanide adjuvants (available from Seppic, Belgium), preferably Montanide ISA-51. Other preferred adjuvants are bacterial DNA based adjuvants, such as adjuvants including CpG oligonucleotide sequences. Yet other preferred adjuvants are viral dsRNA based adjuvants, such as poly I:C. GM-CSF and imidazoquinolines are also examples of preferred adjuvants.

The adjuvant is most preferably a Montanide ISA adjuvant. The Montanide ISA adjuvant is preferably Montanide ISA 51 or Montanide ISA 720.

In Goding, Monoclonal Antibodies: Principles & Practice (2nd edition, 1986) at pages 61-63 it is also noted that, when an antigen of interest is of low molecular weight, or is poorly immunogenic, coupling to an immunogenic carrier is recommended. A polypeptide of the invention may therefore be coupled to a carrier. A carrier may be present independently of an adjuvant. The function of a carrier can be, for example, to increase the molecular weight of a polypeptide fragment in order to increase activity or immunogenicity, to confer stability, to increase the biological activity, or to increase serum half-life. Furthermore, a carrier may aid in presenting the polypeptide or fragment thereof to T-cells. Thus, in the composition, the polypeptide may be associated with a carrier such as those set out below. The carrier may be any suitable carrier known to a person skilled in the art, for example a protein or an antigen presenting cell, such as a dendritic cell (DC). Carrier proteins include keyhole limpet hemocyanin, serum proteins such as transferrin, bovine serum albumin, human serum albumin, thyroglobulin or ovalbumin, immunoglobulins, or hormones, such as insulin or palmitic acid. Alternatively the carrier protein may be tetanus toxoid or diphtheria toxoid. Alternatively, the carrier may be a dextran such as sepharose. The carrier must be physiologically acceptable to humans and safe.

If the composition comprises an excipient, it must be ‘pharmaceutically acceptable’ in the sense of being compatible with the other ingredients of the composition and not deleterious to the recipient thereof. Auxiliary substances, such as wetting or emulsifying agents, pH buffering substances and the like, may be present in the excipient. These excipients and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Formulation of a suitable composition can be carried out using standard pharmaceutical formulation chemistries and methodologies all of which are readily available to the reasonably skilled artisan. Such compositions may be prepared, packaged, or sold in a form suitable for bolus administration or for continuous administration. Injectable compositions may be prepared, packaged, or sold in unit dosage form, such as in ampoules or in multi-dose containers optionally containing a preservative. Compositions include, but are not limited to, suspensions, solutions, emulsions in oily or aqueous vehicles, pastes, and implantable sustained-release or biodegradable formulations. In one embodiment of a composition, the active ingredient is provided in dry (for e.g., a powder or granules) form for reconstitution with a suitable vehicle (e.g., sterile pyrogen-free water) prior to administration of the reconstituted composition. The composition may be prepared, packaged, or sold in the form of a sterile injectable aqueous or oily suspension or solution. This suspension or solution may be formulated according to the known art, and may comprise, in addition to the active ingredient, additional ingredients such as the adjuvants, excipients and auxiliary substances described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. Alternatively, the active ingredients of the composition may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

Formulations of the compositions described herein may be prepared by any method known or hereafter developed in the art. In general, such preparatory methods include the step of bringing the active ingredient into association with an excipient and/or one or more other accessory ingredients, and then, if necessary and/or desirable, dividing, shaping and/or packaging the product into a desired single- or multi-dose unit. Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

mRNA cancer vaccines may be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with mRNA cancer vaccines (e.g., for transplantation into a subject), hyaluronidase, nanoparticle mimics and combinations thereof.

The mRNA and/or compositions disclosed herein may include stabilising elements. Naturally-occurring eukaryotic mRNA molecules have been found to contain stabilizing elements, including, but not limited to the 5′ and 3′ UTRs, the 5′ cap and the 3′ tail discussed elsewhere in this document. Other stabilizing elements that may be included in mRNA as disclosed herein may include for instance a histone stem-loop. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The mRNA may have one or more AU-rich sequences removed. Such sequences may be destabilising. The RNA vaccine may or may not contain a enhancer and/or promoter sequence, which may be modified or unmodified or which may be activated or inactivated.

The mRNA cancer vaccines disclosed herein may be formulated in lipid nanoparticles having a diameter from about 10 to about 200 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 50 to about 150 nm, about 50 to about 200 nm, about 60 to about 70 nm, about 60 to about 80 nm, about 60 to about 90 nm, about 60 to about 100 nm, about 60 to about 150 nm, about 60 to about 200 nm, about 70 to about 80 nm, about 70 to about 90 nm, about 70 to about 100 nm, about 70 to about 150 nm, about 70 to about 200 nm, about 80 to about 90 nm, about 80 to about 100 nm, about 80 to about 150 nm, about 80 to about 200 nm, about 90 to about 100 nm, about 90 to about 150 nm, and/or about 90 to about 200 nm.

The lipid nanoparticles may have a diameter from about 10 to 500 nm. In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

The lipid nanoparticle may be a limit size lipid nanoparticle as described in International Patent Publication No. WO 2013/059922, the contents of which are herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may comprise a polyethylene glycol-lipid such as, but not limited to, DLPE-PEG, DMPE-PEG, DPPC-PEG and DSPE-PEG.

The RNA vaccines may be delivered, localized and/or concentrated in a specific location using the delivery methods described in International Patent Publication No. WO 2013/063530, the contents of which are herein incorporated by reference in its entirety. As a non-limiting example, a subject may be administered an empty polymeric particle prior to, simultaneously with or after delivering the RNA vaccines to the subject. The empty polymeric particle undergoes a change in volume once in contact with the subject and becomes lodged, embedded, immobilized or entrapped at a specific location in the subject.

The lipid nanoparticle composition may comprise a cationic lipid, a PEG-modified lipid, a sterol and a non-cationic lipid. The lipid nanoparticle composition may comprise a molar ratio of about 20-60% cationic lipid: 5-25% non-cationic lipid: 25-55% sterol; and 0.5-15% PEG-modified lipid.

### Methods of Use

The polypeptide, polynucleotide or composition of the invention, or a combination thereof may be used in a method of treating or preventing a disease or condition in a subject.

The polypeptide, polynucleotide or composition of the invention, or combination thereof may be used in the manufacture of a medicament for use in a method of treating or preventing a disease or condition in a subject. The method may comprise administering to the said subject the said polypeptide, the said polynucleotide, the said composition, or the said combination. Administration may be of a therapeutically or prophylactically effective quantity of the said polypeptide, the said polynucleotide, the said composition, or the said combination to a subject in need thereof.

The disease or condition may be characterized at least in part by inappropriate or excessive immune suppressive function of TGFb-1. The disease or condition may be a cancer, preferably a cancer which expresses TGFb-1 and/or which is associated with inappropriate or excessive immune suppressive function of TGFb-1. The cancer may be esophageal cancer or urothelial cancer. The cancer may be colorectal carcinoma, gastric cancer, head and neck cancer, melanoma, non-small-cell lung carcinoma (NSCLC) or ovarian cancer. The cancer may be breast cancer, cervical cancer, liver cancer, or pancreatic cancer. The cancer may be a tumor.

The method may comprise simultaneous or sequential administration with an additional cancer therapy. The additional cancer therapy may be a bi-specific inhibitor of TGFb (e.g. TGFb-1) and PD-L1. Said bi-specific inhibitor may be capable of simultaneously binding to, and/or inhibiting the activity of, TGFb and PD-L1. Said bi-specific inhibitor may be a fusion protein comprising an anti-TGFb portion and an anti-PD-L1 portion, optionally wherein the anti-PD-L1 portion comprises or consists of anti-PD-L1 antibody and/or the anti-TGFb portion comprises or consists of a receptor for TGFb or a portion thereof, such as TGFb receptor II or portion thereof.

The additional cancer therapy may be selected from a cytokine therapy, a T-cell therapy, an NK therapy, an immune system checkpoint inhibitor, chemotherapy, radiotherapy, immunostimulating substances, gene therapy, or an antibody.

The antibody may be Abagovomab, Abciximab, Actoxumab, Adalimumab, Adecatumumab, Afelimomab, Afutuzumab, Alacizumab pegol, ALD518, Alemtuzumab, Alirocumab, Altumomab pentetate, Amatuximab, Anatumomab mafenatox, Anrukinzumab, Apolizumab, Arcitumomab, Aselizumab, Atinumab, Atlizumab (=tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Elotuzumab Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Obinutuzumab, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab (=tremelimumab), Tildrakizumab, Tigatuzumab, TNX-650, Tocilizumab (=atlizumab), Toralizumab, Tositumomab, Tralokinumab, Trastuzumab, TRBS07, Tregalizumab, Tremelimumab Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab Vesencumab, Visilizumab, Volociximab, Vorsetuzumab mafodotin, Votumumab, Zalutumumab, Zanolimumab, Zatuximab, Ziralimumab or Zolimomab aritox.

Preferred antibodies include Natalizumab, Vedolizumab, Belimumab, Atacicept, Alefacept, Otelixizumab, Teplizumab, Rituximab, Ofatumumab, Ocrelizumab, Epratuzumab, Alemtuzumab, Abatacept, Eculizumab, Omalizumab, Canakinumab, Meplizumab, Reslizumab, Tocilizumab, Ustekinumab, Briakinumab, Etanercept, Inlfliximab, Adalimumab, Certolizumab pegol, Golimumab, Trastuzumab, Gemtuzumab, Ozogamicin, Ibritumomab, Tiuxetan, Tostitumomab, Cetuximab, Bevacizumab, Panitumumab, Denosumab, Ipilimumab, Brentuximab and Vedotin.

Particularly preferred antibodies that may be used in the method of the invention include: daratumumab, nivolumab, pembrolizumab, avelumab, rituximab, trastuzumab, pertuzumab, alemtuzumab, cetuximab, panitumumab, tositumomab and of atumumab.

The additional cancer therapy may be selected from the group consisting of Actimide, Azacitidine, Azathioprine, Bleomycin, Carboplatin, Capecitabine, Cisplatin, Chlorambucil, Cyclophosphamide, Cytarabine, Dauno-rubicin, Docetaxel, Doxifluridine, Doxorubicin, Epirubicin, Etoposide, Fludarabine, Fluor-ouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine.

A polypeptide of the invention, a polynucleotide of the invention and/or a composition of the invention may also be used in a method of stimulating TGFb-1-selective T cells, such as CD4⁺ and/or CD8⁺ T-cells, comprising contacting cells with the said polypeptide and/or said composition. The method may be conducted ex vivo. The cells may be present in a sample taken from a healthy subject or from a cancer patient, such as in a tumour sample. The TGFb-1 selective T cells may display low cross reactivity towards TGFb-2 and TGFb-3. The reactivity of the TGFb-1 selective T cells may be compared with the reactivity of TGFb-1 specific T cells which are contacted with a corresponding polypeptide from TGFb-2 or TGFb-3. The reactivity of the TGFb-1 selective T cells to cells expressing and/or presenting a polypeptide of TGFb-1 may be compared with the reactivity of TGFb-1 specific T cells to cells expressing and/or presenting a corresponding polypeptide from TGFb-2 or TGFb-3. The reactivity of TGFb-1 selective T cells may be measured by methods apparent to persons skilled in art, for example IFNγ ELISPOT assay.

A polypeptide of the invention, a polynucleotide of the invention, and/or a composition of the invention may also be used in a method of modulating the tumor microenvironment (TME) of a subject. TGFb-1 may be highly expressed in the TME of most cancer types, for example in colorectal carcinoma, esophageal squamous cell carcinoma, gastric cancer, head and neck cancer, melanoma, NSCLC, ovarian cancer and urothelial carcinoma. In particular, TGFb-1 may be expressed by various cell types in the TME, for example cancer associated fibroblasts (CAFs), CD8⁺ T cells, CD4⁺ T cells, regulatory CD4⁺ T cells, exhausted CD8⁺ T cells, M1 tumor associated macrophages (M1_TAM), M2 tumor associated macrophages (M2_TAM), myeloid antigen presenting cells (APCmye) and other cells. The method comprises administering to a subject the said polypeptide, the said polynucleotide, the said composition, or the said combination. The polypeptides of the invention are capable of eliciting a TGFb-1 selective T cell response, so administration of a polypeptide and/or composition of the invention comprising at least one polypeptide of the invention may be used to modulate the TME of a subject suffering from cancer. Modulating the TME may comprise enhancing T cell infiltration in the TME, for example enhancing CD4⁺ T cell infiltration in the TME.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### EXAMPLES

### Example 1—Materials and Methods

### Peptides

Peptides were synthesized by standard methods and provided dissolved in DMSO to obtain a stock concentration of 5 or 10 mM. The sequences of the peptides used in these experiments are shown in the section entitled “SEQUENCES”). Peptides are described by SEQ ID NO, by name, or by reference to the start and end positions of each peptide sequence within the amino acid sequence of the full length precursor of human TGFb. Each designation may be used interchangeably, as indicated in the table set out in the SEQUENCES section below. For example, the peptide of SEQ ID NO: 7 may alternatively be referred to by the name Pep01-1, or may alternatively be referred to as TGFb-1112-151 (given a start position of 112 and end position of 151). The intended reference in each case will be clear from the context.

### In Vitro ELISPOT Assay

For in vitro ELISPOT, peripheral blood monocytic cells (PBMCs) from healthy donors were pulsed with 20 μM of TGFb-derived peptides and 20 U/ml IL-2 in 24-well plates for 7 days before being used in an ELISPOT assay. The cells were placed in 96-well nitrocellulose ELISPOT plates (MultiScreen IP Filter Plate, MSIPN4W50; Millipore) pre-coated with interferon gamma (IFNγ) capture antibody (Mabtech). TGFb peptides are added to a final concentration of 5 μM, control stimulation (DMSO) added to control wells and plates are incubated at 37° C. for 16-20 hours. After the incubation the cells were washed off and secondary biotinylated Ab (Mabtech) was added for 2 hours at room temperature. Unbound secondary antibody was washed off and streptavidin conjugated alkaline phosphatase (AP) (Mabtech) was added for 1 hour at room temperature. Unbound conjugated enzyme was washed off and the assay was developed by adding BCIP/NBT substrate (Mabtech). Developed ELISPOT plates were analysed on CTL ImmunoSpot S6 Ultimate-V analyzer using Immunospot software v5.1.

### Murine Tumor Study

For the MC38 tumor study, female C56/BL6 mice (Tacomic) were vaccinated with 100 μg of peptide (SEQ ID: 31 or SEQ ID NO: 34) prepared in DMSO and diluted in water to a total volume of 50 μl (per injection) before mixing with an equal volume of Montanide ISA51 VG ST adjuvant to form an emulsion. The vaccine formulation was administered subcutaneously (s.c.) at the tail base on Days 0, 7, and 14. MC38 tumor cells (2e5 per injection) were administered s.c. in the flank on day 0. Every 3 to 4 days, tumors were measured with a Vernier caliper and the tumor volume was calculated using the formula: V=L×W2/2, where V is tumor volume, L is the length of tumor (longer axis), and W is the width of the tumor (shorter axis).

### Analysis of Tumor Microenvironment (TME) by Flow Cytometry

Freshly isolated tumors were dissociated by collagenase digestion to yield a single cell suspension for flow cytometry analysis. Approximately 1 million cells were stained for analysis by flow cytometry using a Symphony Al flow cytometer (BD, Becton Dickinson) with the following antibodies, anti-mouse LAP BV421 (BD, 565638), anti-mouse CD4 BV605 (BD, 743156) and anti-mouse CD8 BV786 (BD, 563332). Data analysis was performed using FlowJo software.

### In Vivo Cytotoxicity Assay

The in vivo cytotoxicity assay was performed by injecting peptide-loaded splenocytes from untreated donor mice into vaccinated mice. Freshly isolated splenocytes were incubated with 5 μM peptide in media (RPMI, 10% Fetal Bovine Serum) for 1 hour at 37 C. Splencoytes were loaded with assay peptide (SLP1_Ib) or control peptide (P53, AIYKKSQHM) separately. The splenocytes were then washed twice in 5 ml PBS+0.5% BSA and then assay peptide loaded cells were labeled with CellTrace Far Red (ThermoFisher) and control peptide loaded cells were labeled with CellTrace Violet (ThermoFisher) using 1/100 of the recommended concentration for 20 minutes at 37 C. The labeled cells were washed twice in PBS before combining equal numbers of cells in PBS at approximately 8 million cells per 200 μl injection volume. Cells in PBS were injected intravenously. Cell killing was analyzed after 18 hours by isolating splenocytes from injected mice and comparing the recovery of FarRed and Violet labeled cells by flow cytometry. Specific killing of assay pulsed splenocytes was calculated as follows:

$$( 1 - [ ( FarRed / Violet ) vaccinated \times ( Violet / FarRed ) injected ] ) \times 100 \% .$$

### Example 2—TGFb-1 Characterization in Tumor Microenvironment of Multiple Solid Cancer Indications

Neogenomics MultiOmyx™ technology was used to evaluate the expression of a panel of 18-biomarkers: ARG1, CD3, CD4, CD8, CD11b, CD68, CD163, FAP, FoxP3, HLADR, IDO1, LAG-3, PanCK, PD-1, PD-L1, SOX-10, TGFb-1, TIGIT and the tumor markers PanCK and SOX10. Proprietary deep learning algorithms were used to classify positive cells for each marker. The data presented here focus on the frequency of positive classified cells and their overlap between various markers. More than 30 regions of interest (ROI) were analyzed for each cancer type.

TABLE 1
cancer types analysed

| Abbreviation | Cancer type |
|---|---|
| CRC | Colorectal |
| ESOPHAGEAL | Esophageal squamous cell carcinoma |
| GASTRIC | Gastric |
| HNN | Head and Neck Cancer |
| MELANOMA | Melanoma (70% Uveal, 30% Cutaneous) |
| NSCLC | Non-small cell lung cancer (Adenocarcinoma) |
| OVARIAN | Ovarian Cancer (Serous Papillary Adenocarcinoma) |
| UROTHELIAL | Urothelial Carcinoma (Bladder Cancer) |

TABLE 2
cell types and markers used to define them

| Label | Description | Markers |
|---|---|---|
| Tumor cell | Tumor cell | PanCK, Sox10 (melanoma) |
| CAF | Cancer Associated Fibroblast | FAP |
| CD8 | CD8⁺ T cell | CD3⁺, CD8⁺ |
| CD4 | CD4⁺ T cell | CD3⁺, CD4⁺ |
| Treg | Regulatory CD4⁺ T cell | CD4⁺, FoxP3⁺ |
| exCD8 | Exhausted CD8⁺ T cell | CD3⁺, CD8⁺, PD-1/TIGIT/ |
| | | LAG-3⁺ |
| M1_TAM | M1 tumor associated | CD68⁺HLADR⁺CD163⁻ |
| | macrophage | |
| M2_TAM | M2 tumor associated | CD68⁺HLADR⁻CD163⁺ |
| | macrophage | |
| APCmye | Myeloid Antigen | CD11b⁺HLADR⁺ |
| | Presenting Cell | |
| Other | No marker expression | None |

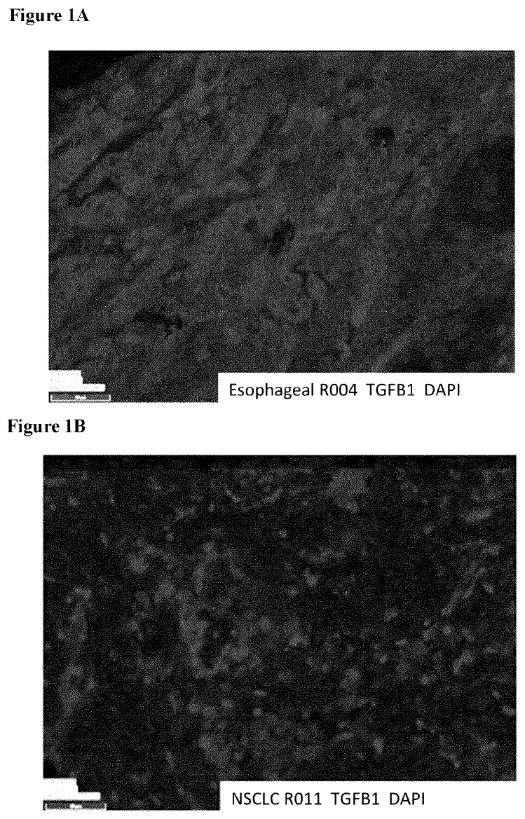

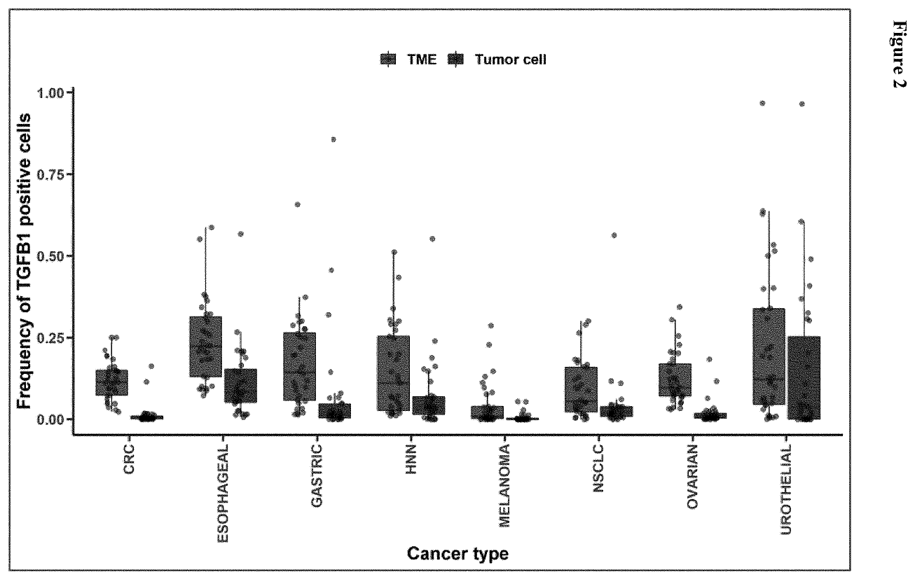

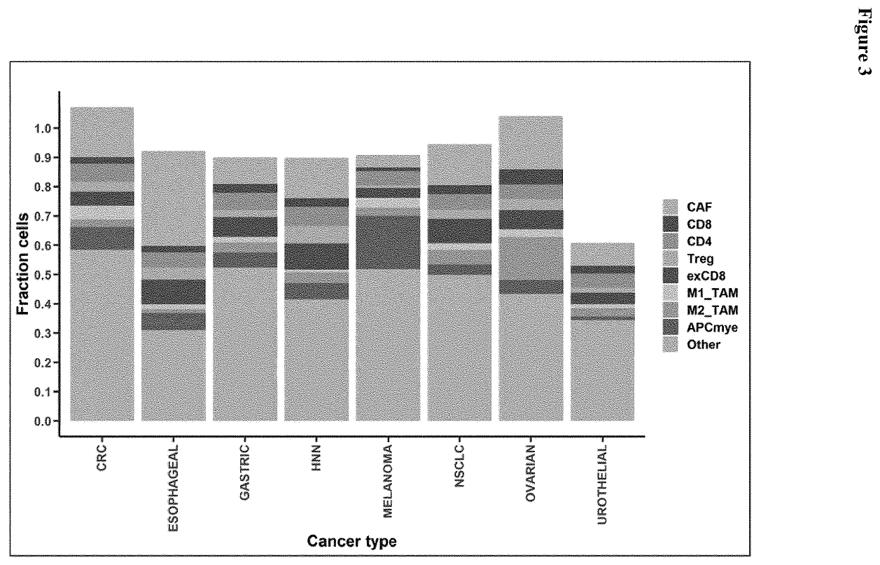

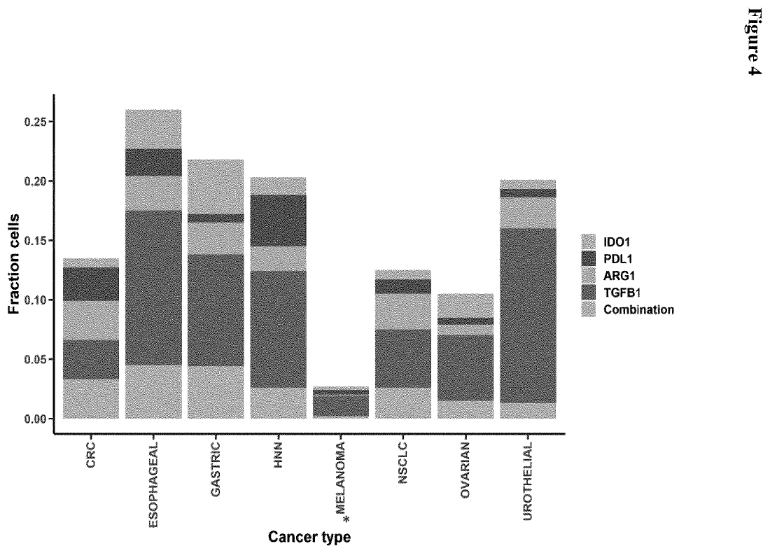

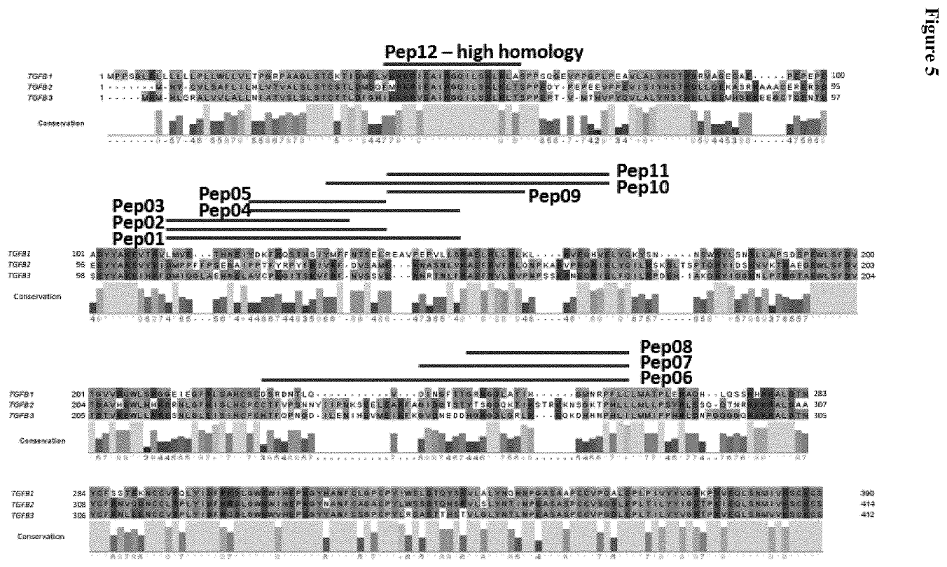

As shown in FIGS. 1-4, TGFb-1 was found to be highly expressed on tumor cells in Esophageal and Urothelial cancers, while TGFb-1 was expressed in the TME of most cancer types. TGFb-1 expressing cells constitute a comparable fraction of tumors as IDO1 and PD-L1, the antigens targeted by IO Biotech's lead therapeutics IO102 and IO103.

### Example 3—Identification and Characterization of TGFb-1-Specific Peptide Antigens

In order to identify peptides with high specificity for the TGFb-1 protein sequence Clustal Omega (www.ebi.ac.uk/Tools/msa/clustalo/) sequence alignment was performed using the UniProt reference sequences (P01137, P61812, and P10600 for TGFb-1, TGFb-2, and TGFb-3, respectively), shown in FIG. 5. The sequence alignment was visualized using Jalview (www.jalview.org/). Five peptides (Pep01-1 to Pep05-1) were selected from low homology region 1 (amino acids 112-151 of SEQ ID NO: 1), three peptides (Pep06-1 to Pep08-1) were selected from low homology region 2 (amino acids 226-260 of SEQ ID NO: 1), and three peptides (Pep09-1 to Pep11-1) were selected to encompass a sequence disclosed in WO 2020/245264 (SEQ ID NO: 205, also referred to as TGFb-15) with intermediate homology. Peptides were selected based on the following criteria: 1) a length equal to or greater than 20 amino acids, 2) avoid any sequential stretch of 8 or more identical or highly similar amino acids, and 3) avoid cysteine residues. A single peptide (Pep12-1) was selected based on a high degree of homology between TGFb-1, TGFb-2, and TGFb-3, and was only used to study TGFb-1-selectivity. The TGFb-1 peptides selected are shown in Table 3, including their percentage sequence identity to corresponding peptides from TGFb-2 and TGFb-3 (see Table 5 for details of corresponding peptides from TGFb-3 and TGFb-3).

TABLE 3
sequence identity

| | | | % identity to | % identity to |
|---|---|---|---|---|
| | | | corresponding | corresponding |
| SEQ | | | TGFb-2 | TGFb-3 |
| ID NO | ID | Sequence | peptide | peptide |
| 7 | Pep01-1 | LMVETHNEIYDKFKQSTHSIYMFFNTSELRE | 18.60 | 23.26 |
| | | AVPEPVLLS | | |
| 8 | Pep02-1 | LMVETHNEIYDKFKQSTHSIYMFFNTSEL | 21.88 | 28.13 |
| 9 | Pep03-1 | LMVETHNEIYDKFKQSTHSIYMFF | 22.22 | 25.93 |
| 10 | Pep04-1 | YDKFKQSTHSIYMFFNTSELREAVPEPVLLS | 16.13 | 19.35 |
| 11 | Pep05-1 | YDKFKQSTHSIYMFFNTSEL | 20.00 | 25.00 |
| 12 | Pep06-1 | DSRDNTLQVDINGFTTGRRGDLATIHGMNRP | 20.00 | 13.73 |
| | | FLLL | | |
| 13 | Pep07-1 | DINGFTTGRRGDLATIHGMNRPFLLL | 34.38 | 24.14 |
| 14 | Pep08-1 | TGRRGDLATIHGMNRPFLLL | 34.62 | 30.43 |
| 15 | Pep09-1 | REAVPEPVLLSRAELRLLRL | 30.00 | 35.00 |
| 16 | Pep10-1 | YMFFNTSELREAVPEPVLLSRAELRLLRLKL | 26.19 | 30.95 |
| | | KVEQHVE | | |
| 17 | Pep11-1 | REAVPEPVLLSRAELRLLRLKLKVEQHVE | 27.27 | 30.30 |
| 28 | Pep12-1 | VKRKRIEAIRGQILSKLRLA | 80.00 | 80.00 |
| 32 | SLP1 Ib | SIYMFFNT | 25.00 | 25.00 |
| 35 | SLP2 Ia | TQYSKVLAL | 66.67 | 55.56 |

### Example 4—Screening for Immune Responses to TGFb-1 Peptides

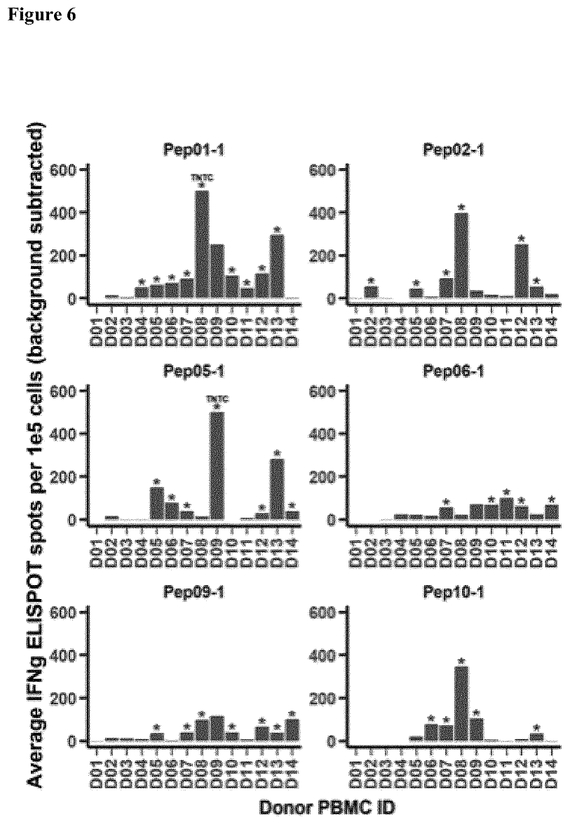

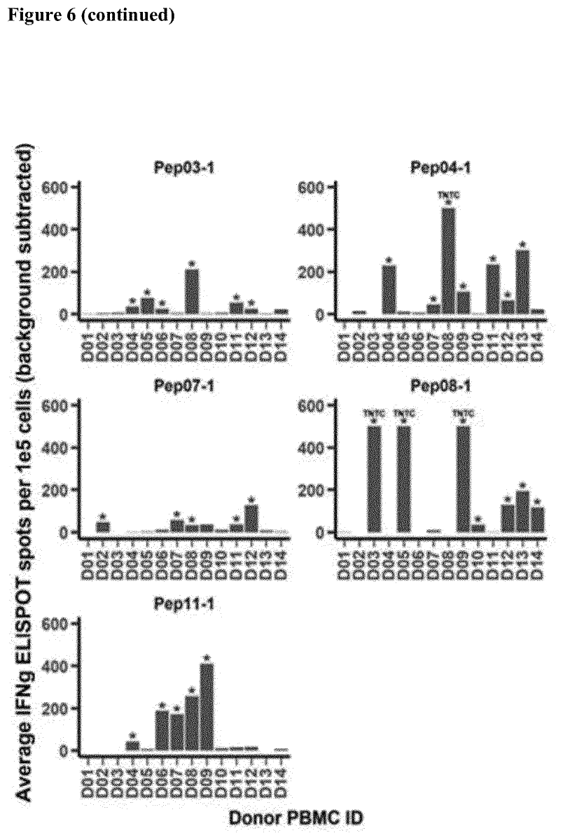

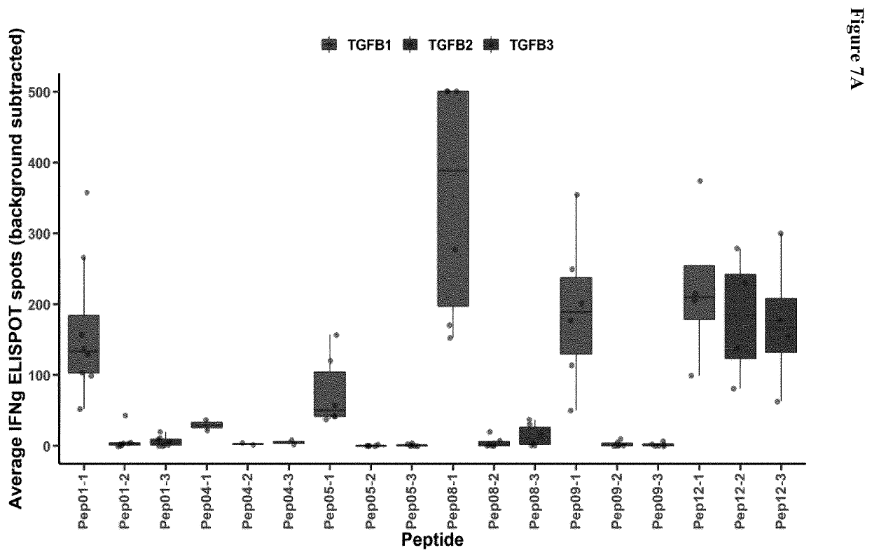

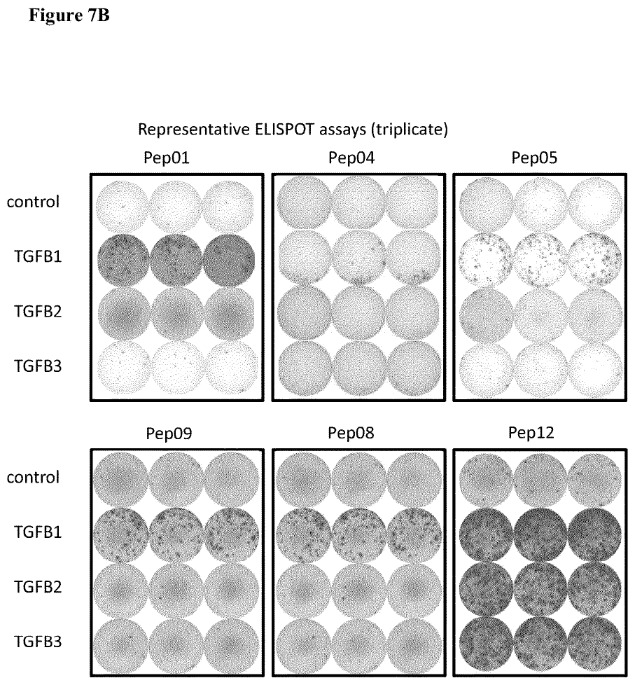

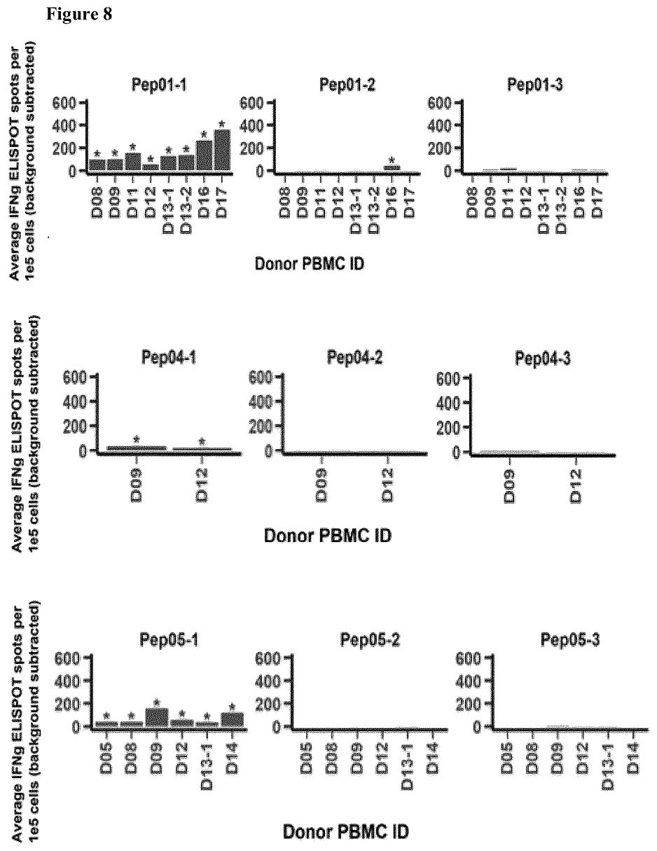

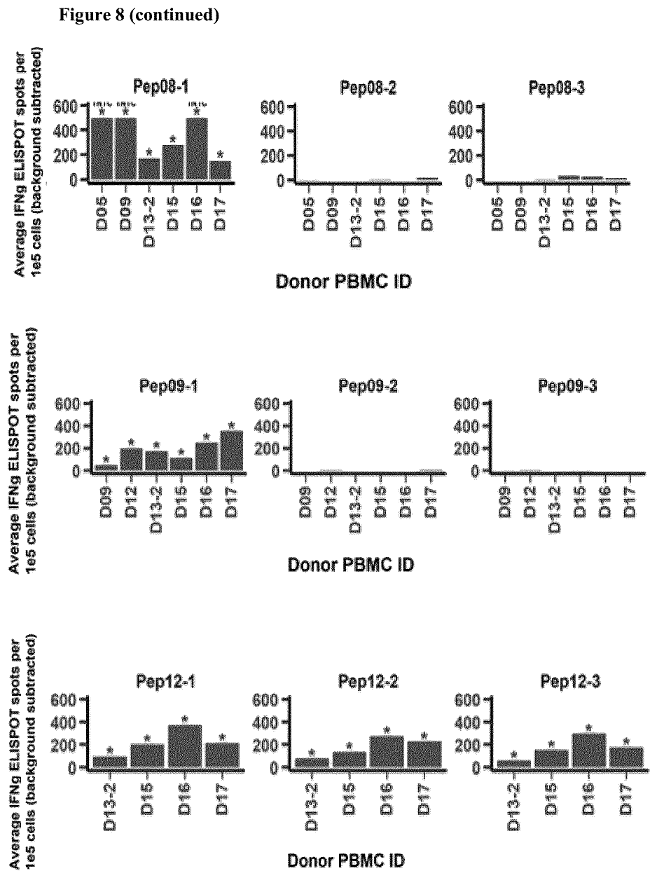

In order to identify whether the low homology peptides selected based on sequence alignment were able to elicit immune responses in humans, the IFNγ ELISPOT assay was used. PBMCs from a total of 14 healthy donors were used to screen for immune responses. PBMCs were exposed to each peptide individually to induce a peptide-specific immune response and proliferation of peptide-specific T cells. Seven days later, the frequency of peptide-specific T cells were assayed by IFNγ ELISPOT. IFNγ ELISPOT identified several peptides that elicited strong (number of spots) and frequent (number of donors) immune responses, as shown in FIG. 6. Five peptides were selected on this basis (see Table 4). These five peptides were subsequently assayed for TGFb-1-selectivity by assaying for cross-reactive immune responses to TGFb-2 and TGFb-3 peptides in the IFNγ ELISPOT assay, as shown in FIG. 7 and FIG. 8. These data show that IFNγ ELISPOT immune responses to TGFb-1 peptides selected for low homology failed to cross-react with the homologous TGFb-2 and TGFb-3 peptides. In contrast, a peptide selected for high homology (Pep12-1), induced immune responses that cross-reacted with homologous TGFb-2 and TGFb-3 peptides (Pep12-2 and Pep12-3, respectively) in a manner that mirrored the magnitude of the TGFb-1-specific response.

TABLE 4
summary of IFNγ ELISPOT screening

| Peptide | Median | Positive Donors | Selected for testing for cross |
|---|---|---|---|
| ID | Spots | (out of 14) | reactivity against TGFb-2/TGFb-3 |
| Pep01-1 | 67 | 9 | Yes |
| Pep02-1 | 28 | 6 | No |
| Pep03-1 | 16 | 6 | No |
| Pep04-1 | 34 | 7 | Yes |
| Pep05-1 | 23 | 7 | Yes |
| Pep06-1 | 25 | 5 | No |
| Pep07-1 | 12 | 5 | No |
| Pep08-1 | 22 | 7 | Yes |
| Pep09-1 | 37 | 7 | Yes |
| Pep10-1 | 8 | 5 | No |
| Pep11-1 | 14 | 5 | No |

Median spots = the median of the background subtracted average spots for each donor across all donors. Positive donors = number of donors (out of 14) that showed a significant responses (Fisher test <0.01, Ratio peptide:control >2, and average spots (background subtracted) >25). Selected peptides were used to test specificity of responses to the TGFb-1 peptide.

Together, these data identify five peptides (Pep01-1, Pep04-1, Pep05-1, Pep08-1, and Pep09-1) that induce strong and frequent immune responses in healthy donors that are selective to TGFb-1.

### Example 5—Functionality of a TGFb-1 Peptide Vaccine in a Mouse Tumor Model

A murine TGFb-1 vaccine was developed based on synthetic long peptides (SLPs) encoding predicted MHC class I and class II epitopes (SEQ ID NOs: 31-36 in Table 1 below). Pre-clinical studies of anti-TGFb therapies often fail to show any impact on tumor growth when administered as a monotherapy. Therefore the goal here primary focused on: 1) developing a vaccine that elicits the most potent immune response consisting of both CD4+ and CD8⁺ T cells; 2) determining any impact on tumor growth; 3) determining any impact on the TME; and 4) developing assays to further characterize vaccine-induced T cell responses.

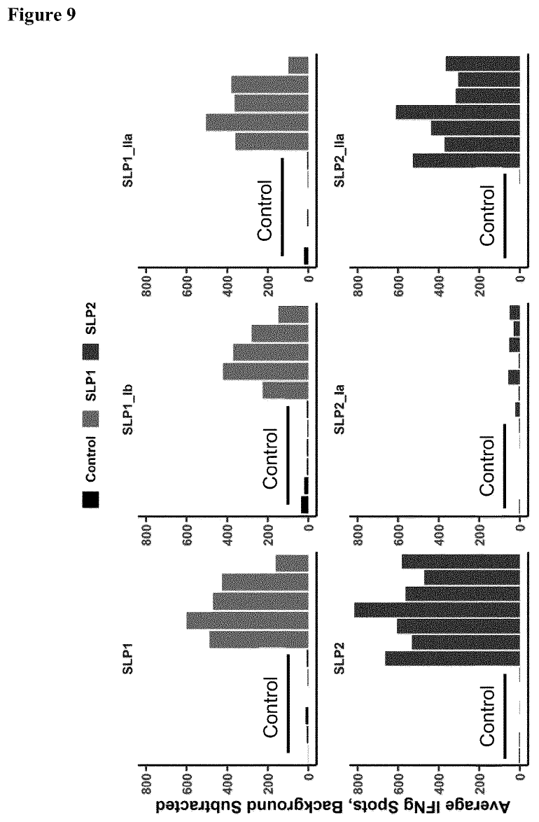

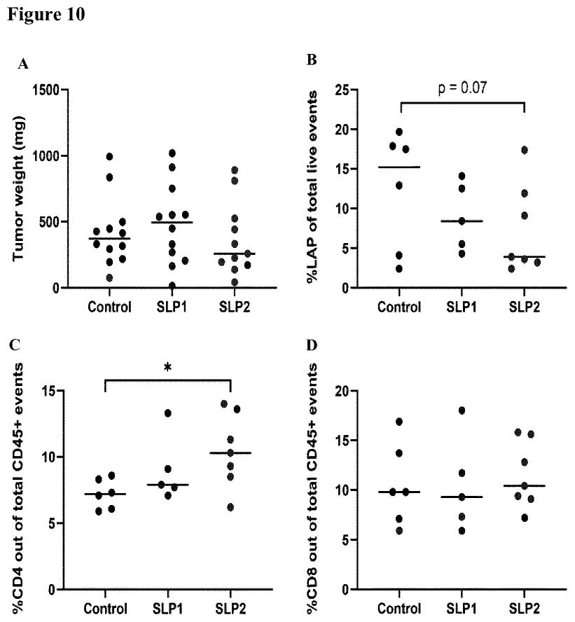

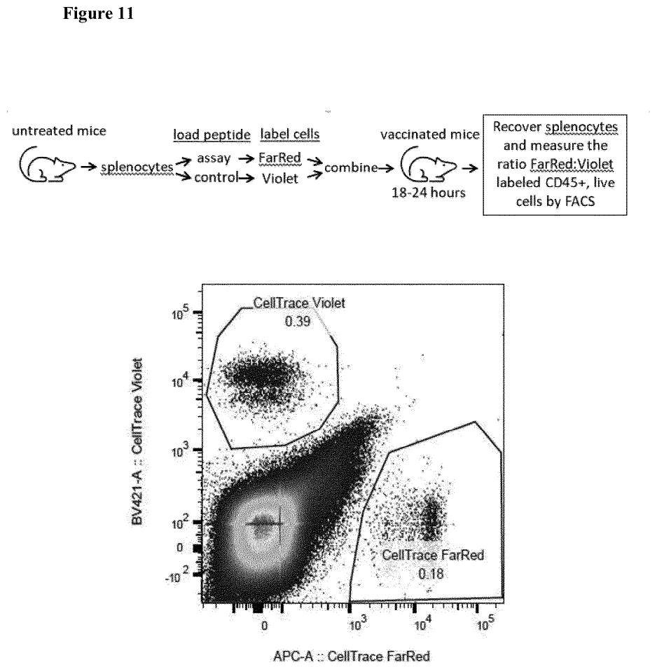

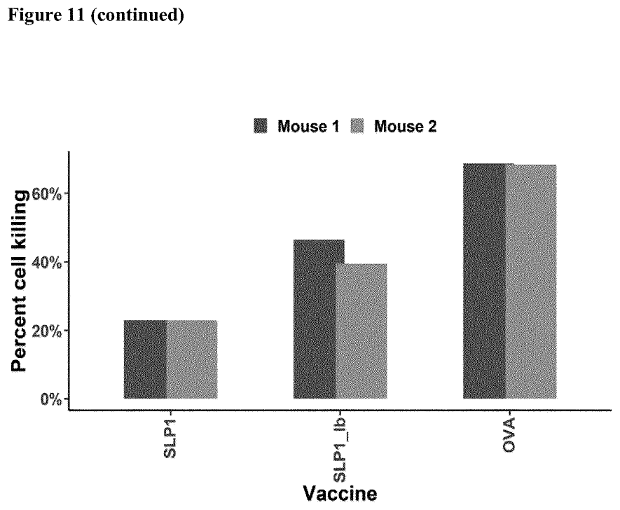

Both SLPs elicited strong immune responses by IFNγ ELISPOT assay, as shown in FIG. 9. Vaccination with SLP1 resulted in recognition of minimal peptides encoding class I and class II epitopes. In contrast, SLP2 mainly induced class II response (although a class I response cannot be ruled out in view of the class I epitopes that were not tested). CD4⁺ T cell infiltration was significantly enhanced in SLP2 vaccinated animals while CD8⁺ T cell infiltration remained unchanged (FIGS. 10C and 10D). An in vivo cytotoxicity assay demonstrated that vaccination with SLP1 results in cytotoxic activity toward cells loaded with the class I peptide antigen SLP1_Ib, as shown in FIG. 11. Notably, cytotoxic activity was higher in animals vaccinated with SLP1_Ib, suggesting superior induction of cytotoxic T cells by the minimal epitope antigen SLP1_Ib compared to SLP1 (FIG. 11). Similar assays are being developed to evaluate TGFb-1 vaccine in mice.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

### SEQUENCES

In Table 5 below, “Start” and “End” indicate the positions within full length human TGFb pre-protein (SEQ ID NO: 1, 2 or 3) unless otherwise indicated.

TABLE 5

| SEQ ID | | | | | |
|---|---|---|---|---|---|
| NO | ID | Sequence | Start | End | Protein |
| 1 | P01137 | MPPSGLRLLLLLLPLLWLLVLTPGRPAAGLSTCKTI | 1 | 390 | TGFb-1 |
| | | DMELVKRKRIEAIRGQILSKLRLASPPSQGEVPPGP | | | |
| | | LPEAVLALYNSTRDRVAGESAEPEPEPEADYYAKEV | | | |
| | | TRVLMVETHNEIYDKFKQSTHSIYMFFNTSELREAV | | | |
| | | PEPVLLSRAELRLLRLKLKVEQHVELYQKYSNNSWR | | | |
| | | YLSNRLLAPSDSPEWLSFDVTGVVRQWLSRGGEIEG | | | |
| | | FRLSAHCSCDSRDNTLQVDINGFTTGRRGDLATIHG | | | |
| | | MNRPFLLLMATPLERAQHLQSSRHRRALDTNYCFSS | | | |
| | | TEKNCCVRQLYIDFRKDLGWKWIHEPKGYHANFCLG | | | |
| | | PCPYIWSLDTQYSKVLALYNQHNPGASAAPCCVPQA | | | |
| | | LEPLPIVYYVGRKPKVEQLSNMIVRSCKCS | | | |
| 2 | P61812 | MHYCVLSAFLILHLVTVALSLSTCSTLDMDQFMRKR | 1 | 414 | TGFb-2 |
| | | IEAIRGQILSKLKLTSPPEDYPEPEEVPPEVISIYN | | | |
| | | STRDLLQEKASRRAAACERERSDEEYYAKEVYKIDM | | | |
| | | PPFFPSENAIPPTFYRPYFRIVRFDVSAMEKNASNL | | | |
| | | VKAEFRVFRLQNPKARVPEQRIELYQILKSKDLTSP | | | |
| | | TQRYIDSKVVKTRAEGEWLSFDVTDAVHEWLHHKDR | | | |
| | | NLGFKISLHCPCCTFVPSNNYIIPNKSEELEARFAG | | | |
| | | IDGTSTYTSGDQKTIKSTRKKNSGKTPHLLLMLLPS | | | |
| | | YRLESQQTNRRKKRALDAAYCFRNVQDNCCLRPLYI | | | |
| | | DFKRDLGWKWIHEPKGYNANFCAGACPYLWSSDTQH | | | |
| | | SRVLSLYNTINPEASASPCCVSQDLEPLTILYYIGK | | | |
| | | TPKIEQLSNMIVKSCKCS | | | |
| 3 | P10600 | MKMHLQRALVVLALLNFATVSLSLSTCTTLDFGHIK | 1 | 412 | TGFb-3 |
| | | KKRVEAIRGQILSKLRLTSPPEPTVMTHVPYQVLAL | | | |
| | | YNSTRELLEEMHGEREEGCTQENTESEYYAKEIHKF | | | |
| | | DMIQGLAEHNELAVCPKGITSKVFRFNVSSVEKNRT | | | |
| | | NLFRAEFRVLRVPNPSSKRNEQRIELFQILRPDEHI | | | |
| | | AKQRYIGGKNLPTRGTAEWLSFDVTDTVREWLLRRE | | | |
| | | SNLGLEISIHCPCHTFQPNGDILENIHEVMEIKEKG | | | |
| | | VDNEDDHGRGDLGRLKKQKDHHNPHLILMMIPPHRL | | | |
| | | DNPGQGGQRKKRALDTNYCFRNLEENCCVRPLYIDF | | | |
| | | RQDLGWKWVHEPKGYYANFCSGPCPYLRSADTTHST | | | |
| | | VLGLYNTLNPEASASPCCVPODLEPLTILYYVGRTP | | | |
| | | KVEQLSNMVVKSCKCS | | | |
| 4 | TGFb-1 | MPPSGLRLLLLLLPLLWLLVLTPGRPAAG | 1 | 29 | TGFb-1 |
| | signal | | | | |
| | peptide | | | | |
| 5 | TGFb-1 | LSTCKTIDMELVKRKRIEAIRGQILSKLRLASPPSQ | 30 | 278 | TGFb-1 |
| | LAP | GEVPPGPLPEAVLALYNSTRDRVAGESAEPEPEPEA | | | |
| | domain | DYYAKEVTRVLMVETHNEIYDKFKQSTHSIYMFFNT | | | |
| | | SELREAVPEPVLLSRAELRLLRLKLKVEQHVELYQK | | | |
| | | YSNNSWRYLSNRLLAPSDSPEWLSFDVTGVVRQWLS | | | |
| | | RGGEIEGFRLSAHCSCDSRDNTLQVDINGFTTGRRG | | | |
| | | DLATIHGMNRPFLLLMATPLERAQHLQSSRHRR | | | |
| 6 | Mature | ALDTNYCFSSTEKNCCVRQLYIDFRKDLGWKWIHEP | 279 | 390 | TGFb-1 |
| | TGFb-1 | KGYHANFCLGPCPYIWSLDTQYSKVLALYNQHNPGA | | | |
| | | SAAPCCVPQALEPLPIVYYVGRKPKVEQLSNMIVRS | | | |
| | | CKCS | | | |
| 7 | Pep01-1 | LMVETHNEIYDKFKQSTHSIYMFFNTSELREAVPEP | 112 | 151 | TGFb-1 |
| | | VLLS | | | |
| 8 | Pep02-1 | LMVETHNEIYDKFKQSTHSIYMFFNTSEL | 112 | 140 | TGFb-1 |
| 9 | Pep03-1 | LMVETHNEIYDKFKQSTHSIYMFF | 112 | 135 | TGFb-1 |
| 10 | Pep04-1 | YDKFKQSTHSIYMFFNTSELREAVPEPVLLS | 121 | 151 | TGFb-1 |
| 11 | Pep05-1 | YDKFKQSTHSIYMFFNTSEL | 121 | 140 | TGFb-1 |
| 12 | Pep06-1 | DSRDNTLQVDINGFTTGRRGDLATIHGMNRPFLLL | 226 | 260 | TGFb-1 |
| 13 | Pep07-1 | DINGFTTGRRGDLATIHGMNRPFLLL | 235 | 260 | TGFb-1 |
| 14 | Pep08-1 | TGRRGDLATIHGMNRPFLLL | 241 | 260 | TGFb-1 |
| 15 | Pep09-1 | REAVPEPVLLSRAELRLLRL | 141 | 160 | TGFb-1 |
| 16 | Pep10-1 | YMFFNTSELREAVPEPVLLSRAELRLLRLKLKVEQH | 132 | 169 | TGFb-1 |
| | | VE | | | |
| 17 | Pep11-1 | REAVPEPVLLSRAELRLLRLKLKVEQHVE | 141 | 169 | TGFb-1 |
| 18 | Pep01-2 | DMPPFFPSENAIPPTFYRPYFRIVREDVSAMEKNAS | 107 | 145 | TGFb-2 |
| | | NLV | | | |
| 19 | Pep04-2 | PPTFYRPYFRIVRFDVSAMEKNASNLV | 119 | 145 | TGFb-2 |
| 20 | Pep05-2 | PPTFYRPYFRIVREDVSAM | 119 | 137 | TGFb-2 |
| 21 | Pep08-2 | TYTSGDQKTIKSTRKKNSGKTPHLLL | 258 | 283 | TGFb-2 |
| 22 | Pep09-2 | EKNASNLVKAEFRVERL | 138 | 154 | TGFb-2 |
| 23 | Pep01-3 | DMIQGLAEHNELAVCPKGITSKVFRFNVSSVEKNRT | 109 | 147 | TGFb-3 |
| | | NLF | | | |
| 24 | Pep04-3 | AVCPKGITSKVFRFNVSSVEKNRTNLF | 121 | 147 | TGFb-3 |
| 25 | Pep05-3 | AVCPKGITSKVFRFNVSSV | 121 | 139 | TGFb-3 |
| 26 | Pep08-3 | DHGRGDLGRLKKQKDHHNPHLIL | 258 | 280 | TGFb-3 |
| 27 | Pep09-3 | EKNRTNLFRAEFRVLRV | 140 | 156 | TGFb-3 |
| 28 | Pep12-1 | VKRKRIEAIRGQILSKLRLA | 41 | 61 | TGFb-1 |
| 29 | Pep12-2 | FMRKRIEAIRGQILSKLKLT | 32 | 51 | TGFb-2 |
| 30 | Pep12-3 | IKKKRVEAIRGQILSKLRLT | 35 | 54 | TGFb-3 |
| 31 | SLP1 | KDISHSIYMFFNTSDIREAVPEPPL | n/a | n/a | Synthetic |
| 32 | SLP1_Ib | SIYMFFNT | 130 | 137 | TGFb-1 |
| 33 | SLP1_IIa | IYMFFNTSDIREAVP | n/a | n/a | Synthetic |
| 34 | SLP2 | SLDTQYSKVLALYNQHNPGASASP | n/a | n/a | Synthetic |
| 35 | SLP2_Ia | TQYSKVLAL | 334 | 342 | TGFb-1 |
| 36 | SLP2_IIa | LALYNQHNPGASASP | n/a | n/a | Synthetic |

1. A polypeptide which is an immunogenic fragment of human transforming growth factor 1 (TGFb-1) and which comprises or consists of a sequence of at least 8 consecutive amino acids of SEQ ID NO: 5.

2. The polypeptide of claim 1, which does not comprise a cysteine residue.

3. The polypeptide of claim 1 or 2, which has low homology and/or low sequence identity to a corresponding polypeptide sequence of human transforming growth factor 2 (TGFb-2) and/or 3 (TGFb-3), optionally wherein:
(a) TGFb-2 has the amino acid sequence of SEQ ID NO: 2 and/or TGFb-3 has the amino acid sequence of SEQ ID NO: 3; and/or
(b) the polypeptide fragment has less than about 80%, 70%, 60%, 50%, 40%, 30%, 25%, or 20% sequence identity to a corresponding polypeptide sequence of TGFb-2 and/or TGFb-3, preferably wherein the polypeptide has less than about 40% sequence identity to a corresponding polypeptide sequence of TGFb-2 and/or TGFb-3.

4. The polypeptide of any one of claims 1-3, which comprises or consists of:
(a) up to 8, 9, 10, 11, 12, 13, 14, 15, 16, 17, 18, 19, 20, 21, 22, 23, 24, 25, 26, 27, 28, 29, 30, 35, or 40 consecutive amino acids of SEQ ID NO: 5; or
(b) at least 8, 9, 10, 11, 12, 13, 14, 15, 16, 17, 18, 19, 20, 21, 22, 23, 24, 25, 26, 27, 28, 29, 30, 35, or 40 consecutive amino acids of SEQ ID NO: 5, preferably wherein the polypeptide comprises or consists of at least 25, 26, 27, 28, 29, or 30 consecutive amino acids of SEQ ID NO: 5, more preferably wherein the polypeptide comprises or consists of at least 30 consecutive amino acids of SEQ ID NO: 5.

5. The polypeptide of any one of claims 1-4, which comprises the amino acid sequence of SEQ ID NO: 32, optionally wherein the polypeptide comprises the amino acid sequence of any one of SEQ ID NOs: 7, 10, or 11.

6. The polypeptide of any one of claims 1-5, which comprises or consists of the amino acid sequence of any one of SEQ ID NOs: 7, 10, 32, 11, 14, or 15, preferably SEQ ID NO: 7 or SEQ ID NO: 10.

7. The polypeptide of any one of claims 1-6, which is capable of stimulating TGFb-1 selective T cells.

8. The polypeptide of claim 7, wherein the TGFb-1 selective T cells have low cross reactivity towards cells expressing and/or presenting polypeptides of TGFb-2 and/or TGFb-3.

9. The polypeptide of claim 8, wherein measurement of cross reactivity comprises comparing the reactivity of the TGFb-1 selective T cells to cells expressing and/or presenting a polypeptide of TGFb-1 with the reactivity of the TGFb-1 selective T cells to cells expressing and/or presenting a corresponding polypeptide from TGFb-2 or TGFb-3, optionally wherein:
(a) the reactivity of the TGFb-1 selective T cells towards cells expressing and/or presenting the corresponding polypeptide from TGFb-2 or TGFb-3 is less than about 50%, 40%, 30%, 20%, 10%, 5%, 1%, or 0.1% of the reactivity of the TGFb-1 selective T cells to the polypeptide of TGFb-1; and/or
(b) cross reactivity is measured by IFNγ ELISPOT assay.

10. A polynucleotide encoding a polypeptide according to any one of claims 1-9, which is optionally comprised within a vector.

11. The polynucleotide of claim 10, which is an mRNA comprising:
(a) an open reading frame (ORF) encoding at least one polypeptide according to any one of claims 1-9;
(b) a 5′ terminal cap at the 5′ end;
(c) a 5′ untranslated region (UTR) which is included 5′ of the ORF;
(d) a 3′ UTR which is included 3′ of the ORF; and
(e) a 3′ tailing sequence at the 3′ end.

12. A composition comprising a polypeptide according to any one of claims 1-9; and/or a polynucleotide according to claim 10 or 11; and optionally an adjuvant.

13. The composition of claim 12 further comprising at least one different polypeptide according to any one of claims 1-9; at least one different polynucleotide according to claim 10 or 11; and/or at least one pharmaceutically acceptable diluent, carrier or preservative.

14. The composition of claim 12 or 13, which comprises an adjuvant selected from the group consisting of bacterial DNA based adjuvants, oil/surfactant based adjuvants, viral dsRNA based adjuvants, imidazoquinolines, and a Montanide ISA adjuvant.

15. The composition of claim 12 or 13, which is formulated in a lipid nanoparticle composition, wherein the composition comprises a polynucleotide and the polynucleotide is an mRNA, optionally wherein the lipid nanoparticle has a mean diameter of 50-200 nm.

16. The composition of any one of claims 12-15, which is a TGFb-1 selective vaccine composition.

17. A method of treating or preventing a disease or condition in a subject, the method comprising administering to the subject a polypeptide as defined in any one of claims 1-9, a polynucleotide as defined in claim 10 or 11, and/or a composition as defined in any one of claims 12-16.

18. The method of claim 17, wherein the disease or condition is:
(a) a cancer, optionally selected from the group consisting of esophageal cancer and urothelial cancer, further optionally selected from the group consisting of colorectal carcinoma, gastric cancer, head and neck cancer, melanoma, non-small-cell lung carcinoma (NSCLC) or ovarian cancer; and/or
(b) characterized at least in part by inappropriate or excessive immune suppressive function of TGFb-1-expressing cells.

19. The method of claim 17 or 18, wherein the disease or condition is cancer and the method further comprises the simultaneous or sequential administration of an additional cancer therapy, preferably an antibody.

20. The method of any one of claims 17-19, wherein the disease or condition is cancer and administering the polypeptide, the polynucleotide and/or the composition stimulates a selective immune response to TGFb-1-expressing cancer cells.

21. The method of any one of claims 17-20, wherein the disease or condition is a tumor and wherein:
(a) the polypeptide, polynucleotide, and/or the composition is capable of modulating the tumor microenvironment (TME), optionally wherein the modulating comprises enhancing infiltration of the TME by T cells, preferably wherein the T cells are CD4⁺ T cells;
(b) the method comprises modulating the TME, optionally wherein the modulating comprises enhancing infiltration of the TME by T cells, preferably wherein the T cells are CD4⁺ T cells; and/or
(c) administering the polypeptide, the polynucleotide and/or the composition stimulates a selective immune response to TGFb-1-expressing cells in the TME.

22. A method of stimulating TGFb-1 selective T cells, the method comprising contacting the T cells with a polypeptide as defined in any one of claims 1-9, a polynucleotide as defined in claim 10 or 11, and/or a composition as defined in any one of claims 12-16.

23. The method of claim 22, wherein the TGFb-1 selective T cells have low cross reactivity towards cells expressing and/or presenting polypeptides of TGFb-2 and/or TGFb-3.

24. The method of claim 23, wherein measurement of cross reactivity comprises comparing the reactivity of the TGFb-1 selective T cells to cells expressing and/or presenting a polypeptide of TGFb-1 with the reactivity of the TGFb-1 selective T cells to cells expressing and/or presenting a corresponding polypeptide from TGFb-2 or TGFb-3, optionally wherein:
(a) the reactivity of the TGFb-1 selective T cells towards cells expressing and/or presenting the corresponding polypeptide from TGFb-2 or TGFb-3 is less than about 50%, 40%, 30%, 20%, 10%, 5%, 1%, or 0.1% of the reactivity of the TGFb-1 selective T cells to the polypeptide of TGFb-1; and/or
(b) cross reactivity is measured by IFNγ ELISPOT assay.


SEQUENCE LISTING (36 sequences)

SEQ ID NO: 1 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 3 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 4 - protein, 29 aa, synthetic construct
MPPSGLRLLLLLLPLLWLLVLTPGRPAAG

SEQ ID NO: 5 - protein, 249 aa, synthetic construct
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 6 - protein, 112 aa, synthetic construct
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 7 - protein, 40 aa, synthetic construct
LMVETHNEIYDKFKQSTHSIYMFFNTSELREAVPEPVLLS

SEQ ID NO: 8 - protein, 29 aa, synthetic construct
LMVETHNEIYDKFKQSTHSIYMFFNTSEL

SEQ ID NO: 9 - protein, 24 aa, synthetic construct
LMVETHNEIYDKFKQSTHSIYMFF

SEQ ID NO: 10 - protein, 31 aa, synthetic construct
YDKFKQSTHSIYMFFNTSELREAVPEPVLLS

SEQ ID NO: 11 - protein, 20 aa, synthetic construct
YDKFKQSTHSIYMFFNTSEL

SEQ ID NO: 12 - protein, 35 aa, synthetic construct
DSRDNTLQVDINGFTTGRRGDLATIHGMNRPFLLL

SEQ ID NO: 13 - protein, 26 aa, synthetic construct
DINGFTTGRRGDLATIHGMNRPFLLL

SEQ ID NO: 14 - protein, 20 aa, synthetic construct
TGRRGDLATIHGMNRPFLLL

SEQ ID NO: 15 - protein, 20 aa, synthetic construct
REAVPEPVLLSRAELRLLRL

SEQ ID NO: 16 - protein, 38 aa, synthetic construct
YMFFNTSELREAVPEPVLLSRAELRLLRLKLKVEQHVE

SEQ ID NO: 17 - protein, 29 aa, synthetic construct
REAVPEPVLLSRAELRLLRLKLKVEQHVE

SEQ ID NO: 18 - protein, 39 aa, synthetic construct
DMPPFFPSENAIPPTFYRPYFRIVRFDVSAMEKNASNLV

SEQ ID NO: 19 - protein, 27 aa, synthetic construct
PPTFYRPYFRIVRFDVSAMEKNASNLV

SEQ ID NO: 20 - protein, 19 aa, synthetic construct
PPTFYRPYFRIVRFDVSAM

SEQ ID NO: 21 - protein, 26 aa, synthetic construct
TYTSGDQKTIKSTRKKNSGKTPHLLL

SEQ ID NO: 22 - protein, 17 aa, synthetic construct
EKNASNLVKAEFRVFRL

SEQ ID NO: 23 - protein, 39 aa, synthetic construct
DMIQGLAEHNELAVCPKGITSKVFRFNVSSVEKNRTNLF

SEQ ID NO: 24 - protein, 27 aa, synthetic construct
AVCPKGITSKVFRFNVSSVEKNRTNLF

SEQ ID NO: 25 - protein, 19 aa, synthetic construct
AVCPKGITSKVFRFNVSSV

SEQ ID NO: 26 - protein, 23 aa, synthetic construct
DHGRGDLGRLKKQKDHHNPHLIL

SEQ ID NO: 27 - protein, 17 aa, synthetic construct
EKNRTNLFRAEFRVLRV

SEQ ID NO: 28 - protein, 20 aa, synthetic construct
VKRKRIEAIRGQILSKLRLA

SEQ ID NO: 29 - protein, 20 aa, synthetic construct
FMRKRIEAIRGQILSKLKLT

SEQ ID NO: 30 - protein, 20 aa, synthetic construct
IKKKRVEAIRGQILSKLRLT

SEQ ID NO: 31 - protein, 25 aa, synthetic construct
KDISHSIYMFFNTSDIREAVPEPPL

SEQ ID NO: 32 - protein, 8 aa, synthetic construct
SIYMFFNT

SEQ ID NO: 33 - protein, 15 aa, synthetic construct
IYMFFNTSDIREAVP

SEQ ID NO: 34 - protein, 24 aa, synthetic construct
SLDTQYSKVLALYNQHNPGASASP

SEQ ID NO: 35 - protein, 9 aa, synthetic construct
TQYSKVLAL

SEQ ID NO: 36 - protein, 15 aa, synthetic construct
LALYNQHNPGASASP